(19) United States
(12) Patent Application Publication
(10) Pub. No.: US 2020/0376130 A1
(43) Pub. Date: Dec. 3, 2020

(54) COMPOUNDS AND METHODS USEFUL IN BRACHYTHERAPY

(71) Applicant: North Carolina State University, Raleigh, NC (US)

(72) Inventors: Jonathan S. Lindsey, Raleigh, NC (US); Hikaru Fujita, Raleigh, NC (US); Yunlong Zhang, Raleigh, NC (US)

(21) Appl. No.: 16/970,442

(22) PCT Filed: Feb. 22, 2019
(86) PCT No.: PCT/US2019/019090

§ 371 Date: Aug. 17, 2020

Related U.S. Application Data
(60) Provisional application No. 62/633,849, filed on Feb. 22, 2018.

(51) Int. Cl. A61K 47/54; A61K 47/55; A61K 47/65

(52) U.S. Cl. CPC A61K 47/549; A61K 47/551; A61K 45/06; A61K 47/65; A61K 47/556

(57) ABSTRACT

Compounds and methods are described herein that are useful in brachytherapy. A compound of the present invention may comprise: a cancer cell targeting agent (e.g., transferrin); a protecting group; a cross-linking moiety; and an enzyme (e.g., a protein, ribozyme, abzyme, or abiological catalyst). Compounds and methods of the present invention may be used for localizing a radioactive compound and/or for creating a self-amplifying response.

### STATEMENT OF PRIORITY

This application claims the benefit, under 35 U.S.C. § 119(e), of U.S. Provisional Application No. 62/633,849, filed on Feb. 22, 2018, the entire contents of which are incorporated by reference herein.

### FIELD

The present invention concerns compounds and methods useful in brachytherapy including compounds and methods for localizing a radioactive compound and/or for creating a self-amplifying response.

### BACKGROUND

Tumors are known to be composed of a diversity of cell types. The diversity arises owing to mutation, genetic instability and hypermutation. The presence of a rich collection of different cell types has presented, and continues to present, significant challenges to traditional chemotherapeutic approaches. The one-target—one-drug model at the heart of chemotherapeutics appears insufficient when there exists a multiplicity of cell types that would require independent targeting. A repertoire of chemotherapeutics aimed at each of the various cell types could constitute a viable approach, yet the identification and development of such a repertoire presents enormous challenges. Numerous therapeutic approaches have been examined to overcome the diversity of cell types in tumors. The objective is to target the therapeutic agent to the diseased tissue while leaving normal cells intact.

Selective targeting of diseased tissues is a longstanding objective in medicine. The field of photodynamic therapy (PDT) relies on selective accumulation or targeting of a diseased tissue with a photosensitizer, illumination of the diseased tissue to activate the photosensitizer, and the presence of molecular oxygen to yield reactive oxygen species by the photoactivated photosensitizer. Cell killing occurs by the reactive oxygen species, which are produced in the presence of the triad of photosensitizer, light and oxygen. In early approaches, PDT relied on selective accumulation of the photosensitizer in diseased tissue, whereas more recent approaches may rely on delivery of the photosensitizer via antibody targeting. The indiscriminate killing of reactive oxygen species is attractive, although the field over which the killing occurs may not encompass metastatic cells that lack antigens or other targeted entities. The “bystander effect” may hence be limited. The light source is typically focused on the sites where tumors are known to be present such as in the mouth or throat, or on surface-accessible sites. Other issues that may limit the scope and utility of PDT include the occurrence of systemic pain, and the requirement to limit exposure by the patient to sunlight or other bright lights for days following treatment. The use of targeted light sources would not be effective for eradicating micrometastases.

Approaches to selective location of therapeutic agents rely on prodrugs, which are non-therapeutic in their native state but upon enzymatic treatment undergo conversion to the drug. In this case, the key design feature is to create prodrugs that are selectively converted to the drug by enzymes that are present, if not uniquely, at least in elevated concentration, in diseased tissue. The prodrug approach has been extended to include antibodies for selective targeting of diseased tissue. Here, an enzyme for the prodrug-to-drug conversion is conjugated to the antibody; the antibody is chosen for selective binding to diseased versus normal tissue. This approach is termed antibody-directed enzyme prodrug therapy (ADEPT).

To selectively kill tumor cells but diminish side effects to normal cells, localization of enzymes at tumor cells affords a number of attractions. For ADEPT, antibody-enzyme conjugates have to maintain a low enzyme level in the blood due to two selectivity limitations. First, only a small portion of the antibody-enzyme conjugate binds at tumor cells, compared with that remaining in the circulation before clearance. Second, the bound antibody-enzyme conjugates can leak back into the blood from the cancer sites (“The First Bagshawe Lecture Towards Generating Cytotoxic Agents at Cancer Sites,” Bagshawe, K. D. Br. J. Cancer 1989, 60, 275-281; and “Antibody-Directed Enzyme Prodrug Therapy (ADEPT) for Cancer,” Sharma, S. K.; Bagshawe, K. D. In Macromolecular Anticancer Therapeutics; Reddy, L. H., Couvreur, P, Eds.; Humana Press: New York, N.Y., 2010; pp 392-406).

A core part of ADEPT is the use of prodrugs, but this is not without limitations. Administration of prodrugs is based on the localization of antibody-enzyme conjugates, which relies on targeting of antibodies to the overexpressed antigens of tumors (“Anti-tumor Effects of Antibody-Alkaline Phosphatase Conjugates in Combination with Etoposide Phosphate,” Senter, P. D.; Saulnier, M. G.; Schreiber, G. J.; Hirschberg, D. L.; Brown, J. P.; Hellstrom, I.; Hellström, K. E. Proc. Natl. Acad. Sci. USA 1988, 85, 4842-4846; and “Construction, Expression, and Activities of L49-sFv-β-Lactamase, a Single-Chain Antibody Fusion Protein for Anticancer Prodrug Activation,” Siemers, N. O.; Kerr, D. E.; Yarnold, S.; Stebbins, M. R.; Vrudhula, V. M.; Hellstrom, I.; Hellstrom, K. E.; Senter, P. D. Bioconjugate Chem. 1997, 8, 510-519). Antigen shedding by tumor cells is a known limitation. Moreover, the activated drugs are still diffusible through the body. One principle of prodrug design is to develop compounds having low cytotoxicity before activation but high cytotoxicity after activation (“Development of a Humanized Disulfide-Stabilized Anti-p185^{HER2} Fv-β-Lactamase Fusion Protein for Activation of a Cephalosporin Doxorubicin Prodrug,” Rodrigues, M. L., et al., Cancer Res. 1995, 55, 63-70; and “Proof of Principle in the Selective Treatment of Cancer by Antibody-Directed Enzyme Prodrug Therapy: The Development of a Highly Potent Prodrug,” Tietze, L. F., et al., Angew. Chem. Int. Ed. 2002, 41, 759-761). A further principle concerns the development of drugs with short half-times (“Antibody Directed Enzymes Revive Anti-Cancer Prodrugs Concept,” Bagshawe, K. D. Br. J. Cancer 1987, 56, 531-532).

Numerous antibodies and antigens are under investigation for ADEPT (Senter et al., Siemers et al., “Characterization of a CC49-Based Single-Chain Fragment-β-Lactamase Fusion Protein for Antibody-Directed Enzyme Prodrug Therapy,” Alderson, R. F., et al., Bioconjugate Chem. 2006, 17, 410-480., and “Development and Activities of a New Melphalan Prodrug Designed for Tumor Selective Activation,” Kerr, D. E., et al., Bioconjugate Chem. 1996, 9, 255-259), but, to our knowledge, to date only one target carcinoembryonic antigen has been employed as a target for an ADEPT therapeutic that has entered the clinic (“Molecular and Functional Characterisation of a Fusion Protein Suited for Tumor Specific Prodrug Activation,” Bosslet, K., et al., J. Cancer 1992, 65, 234-238).

The presence of diverse cell types, and the limitations of surgery (e.g., poorly defined margins between diseased and normal tissues; precious but unidentifiable nerves pervading the tumor) has led to the development of brachytherapy. In brachytherapy, the surgeon implants radioactive pellets or needles at one or more sites in the tumor. In this manner, a radiation field is achieved that spans multiple cells surrounding the implanted radioactive entity. The challenge for the surgeon is to implant the entities in a uniform manner throughout the tumor. Brachytherapy is applied to treat certain tumors (e.g., colon, prostate) but is not useful in many tumors given surgical limitations. Brachytherapy with surgically implanted needles or pellets is not applicable for treatment of micrometastases.

To overcome some of the limitations of ADEPT, particularly the diffusion of the drug in the body, an approach dubbed enzyme-mediated insolubilization therapy (EMIT) or enzyme-mediated cancer imaging and therapy (EMCIT) was developed; the approaches differ only in name and hereafter are termed EMCIT. The approach is summarized in the following: “Bystander Effect Produced by Radiolabeled Tumor Cells in vivo,” Xue, L, Y., et al., Proc. Natl. Acad. Sci. 2002, 99, 13765-13770; “Synthesis and Biologic Evaluation of Radioiodinated Quinazolinone Derivative for Enzyme-Mediated Insolubilization Therapy,” Ho, N. -H., et al., Bioconjugate Chem. 2002, 13, 357-364; “In Silico Design, Synthesis, and Biological Evaluation of Radioiodinated Quinazolinone Derivatives for Alkaline Phosphatase-Mediated Cancer Diagnosis And Therapy,” Chen, K., et al., Mol. Cancer Ther. 2006, 5, 3001-3013; “Effect of Chemical, Physical, and Biologic Properties of Tumor-Targeting Radioiodinated Quinazolinone Derivative,” Wang, K., et al., Bioconjugate Chem. 2007, 18, 754-764, “Molecular-Docking-Guided Design, Synthesis, and Biologic Evaluation of Radioiodinated Quinazolinone Prodrugs,” Chen, K., et al., J. Med. Chem. 2007, 50, 663-673, “Computational Modeling and Experimental Evaluation of a Novel Prodrug for Targeting the Extracellular Space of Prostate Tumors,” Pospisil, P, et al., Cancer Res. 2007, 67, 2197-2204, “Novel Prodrugs for Targeting Diagnostic and Therapeutic Radionuclides to Solid Tumors,” Kassis, A. I., et al., Molecules 2008, 13, 391-404, “Solid-Tumor Radionuclide Therapy Dosimetry: New Paradigms in View of Tumor Microenvironment and Angiogenesis,” Zhu, X., et al., Med. Phys. 2010, 37, 2974-2984, “Computational and Biological Evaluation of Quinazolinone Prodrug for Targeting Pancreatic Cancer,” Pospisil, P., et al., Chem. Biol. Drug Des. 2012, 79, 926-934, and “Computational and Biological Evaluation of Quinazolinone Prodrug for Targeting Pancreatic Cancer,” Pospisil, P.; Kassis, A. I. In Molecular Diagnostics and Treatment of Pancreatic Cancer, Azmi, F., Eds; Academic Press: San Diego, Calif., 2014, pp 385-403, and U.S. Pat. Nos. 7,514,067; 8,168,159; 8,394,953; 8,603,437; 9,186,425; and 9,320,815. In general, the approach entails administration of a water-soluble radiolabeled agent containing a water-solubilizing group that is cleaved by an enzyme; the enzyme is abundant in the extracellular space of the tumor. The enzymatic cleavage causes the radiolabeled agent to undergo conversion to a water-insoluble compound, which precipitates and remains immobile at the site of cleavage in the extracellular space of the tumor. The substrate almost exclusively employed was a radioiodinated quinazolinyl-phosphoester (Xue et al., Wang et al., Chen et al., Pospisil et al. 2012, and Pospisil et al., 2014). The enzyme is typically expressed (i) on the exterior surface of tumor cells, and (ii) in abundance relative to the amount on non-tumor cells.

Another approach to the treatment of metastatic cancer is described in “A Proposal for a New Direction to Treat Cancer,” Rose, S. J. Theor. Biol. 1998, 195, 111-128, hereinafter referred to as the “Rose approach”. The Rose approach entails targeted molecular brachytherapy yet is quite distinct from EMCIT in (i) the nature of the targeting, (ii) the delivery of the enzyme, (iii) and the immobilization of the enzyme in the target tissue. Four intravenous infusions (Steps) are administered sequentially over a period of about 7-10 days. The Rose approach has also been termed the Oncologic approach (“Targeted Molecular Brachytherapy,” Mayers, G. L. Drug Dev. Res. 2006, 67, 94-106). The four steps are described as follows.

The Step 1 agent is a compound containing a cancer-targeting agent (CTA), two or more protected cross-linking agents, and an irreversible enzyme inhibitor or covalently binding ligand (termed LIG, e.g., Loracarbef). Upon uptake by cells via an endocytosing receptor and entering the endosomal pathway, the protecting groups are cleaved by a native endosomal or lysosomal enzyme, thereby unveiling the reactive cross-linking agents. An immobile structure in which LIG is embedded (platform-LIG) is created in the endosome and/or lysosome by self-reaction of the cross-linking agents. The Step 1 agent in the circulation is allowed to clear.

The Step 2 agent is a relatively non-toxic low dose of a currently approved chemotherapy agent (perhaps 25% of a normal dose). Administration of the Step 2 agent kills and breaks open the hyper-sensitive fraction of cancer cells (perhaps 50%) while sparing normal cells, releasing the platform-LIG into the tumor extracellular space (ECS); such platform-LIG present in any normal cells is retained inside the normal cells.

The Step 3 agent is a bispecific reagent, one half of which binds specifically and irreversibly to LIG, and the other half of which is a non-mammalian enzyme. Administration of the Step 3 agent, which is too large to enter cells, results in binding of the non-mammalian enzyme to the platform-LIG in the ECS.

The Step 4 agent is a soluble radioisotopically substituted compound that is converted to an insoluble form upon action of the non-mammalian enzyme. Any soluble radioisotope reagent not converted to insoluble form within the tumors is rapidly excreted, minimizing toxicity to normal tissues.

The Rose approach has practical limitations. In the only literature report describing the approach (Mayers, G. L.), (i) the Step 1 agent was found to give rise to a polyindigo platform upon treatment in solution with the appropriate enzyme or in cells in culture, (ii) the “LIG” unit on the platform formed in solution or in cells in culture was accessible upon treatment with a corresponding mutant β-lactamase (step 3 Agent), and (iii) treatment of mice bearing a syngeneic mammary tumor with a radiolabeled step 1 agent gave rise to a larger quantity of radiolabel in tumor necrotic tissue than in tumor tissue. In addition, the requirement to administer four agents to a patient in itself is not prohibitive as cancer patients often receive multiple drugs, but the requirement for four successive agents posed problems.

Accordingly, new therapies and methods are needed.

### SUMMARY

A first aspect of the present invention is directed to a compound (e.g., first agent) comprising: a cancer cell targeting agent (e.g., transferrin); a protecting group; a cross-linking moiety; and an enzyme (e.g., a protein, ribozyme, abzyme, or abiological catalyst).

Another aspect of the present invention is directed to a method of treating a subject having a solid tumor and/or reducing the size of a solid tumor in a subject, the method comprising: administering a first agent comprising an enzyme (e.g., a protein, ribozyme, abzyme, or abiological catalyst) to the subject; administering a second agent to the subject, wherein the second agent comprises an anti-cancer agent (e.g., a chemotherapeutic agent); and administering a radionuclide-derivatized compound to the subject, wherein the radionuclide-derivatized compound comprises a substrate for the enzyme, thereby treating the subject having the solid tumor and/or reducing the size the solid tumor in the subject.

A further aspect of the present invention is directed to a method of treating a subject having a solid tumor and/or reducing the size a solid tumor in a subject, the method comprising: localizing a first agent comprising an enzyme (e.g., a protein, ribozyme, abzyme, or abiological catalyst) in a cancer cell in the subject; releasing the enzyme from the cancer cell into the extracellular fluid (e.g., into the extracellular space of the tumor); and administering a radionuclide-derivatized compound to the subject, wherein the radionuclide-derivatized compound is converted by the enzyme from a soluble form to a less soluble (e.g., an insoluble) form, thereby treating the subject having the solid tumor and/or reducing the size the solid tumor in the subject.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1 is a schematic illustration of an example method according to embodiments of the present invention.

FIG. 2 is a schematic illustration of example cross-linking moieties (X) in Formula I or II and their example reactions according to embodiments of the present invention.

FIG. 3 is a schematic illustration of hetero crosslinking according to embodiments of the present invention.

FIGS. 4-10 each show an example generic structure of Formula I and a specific example first agent according to embodiments of the present invention.

FIGS. 11 and 12 each show an example generic structure of Formula II and a specific example first agent according to embodiments of the present invention.

FIG. 13 is a schematic showing PEGylation of a lysine residue via reactive approaches and species: (a) N-Hydroxysuccinimidyl ester, (b) succinimidyl carbonate, (c) squaraine, (d) chlorotriazine, (e) tosylate displacement, and (f) reductive amination.

FIG. 14 is a schematic showing PEGylation of a cysteine residue via reactive approaches and species: (a) maleimide, (b) vinylsulfone, (c) iodocarbonyl compound, and (d) 2-pyridyl disulfide.

FIG. 15 is a schematic showing PEGylation of certain amino acid residues.

FIG. 16 is a schematic showing general methods for the modification of an enzyme and/or a carrier protein to attach a PEG-X-PG moiety. BSA is an example carrier protein. A lysine residue provides an example modification site for the enzyme or BSA. Method 1: 1-step modification using a PEGylating agent possessing the X-PG group. Method 2: functional group conversion followed by the attachment of PEG-X-PG. Method 3: PEGylation followed by attachment of X-PG. Here, the “n” displayed may be non-identical.

FIG. 17 is a schematic showing general procedures for single enzyme nanogel (SEN) formation. Top: an original 2-step procedure. Bottom: a modified 1-step procedure. Here, the “n” displayed may be non-identical.

FIG. 18 is a schematic showing general procedures to introduce PEG-X-PG and CTA-PEG to SENs in a statistical manner. Here, the “n” displayed may be non-identical.

FIG. 19 is a schematic showing general procedures for preparation of a SEN-CTA conjugate from an ENZ-CTA (prepared in a rational manner, with a 1:1 ratio of ENZ and CTA) and attachment of PEG-X-PG groups in a statistical manner affording product mixtures. Here, the “n” displayed may be non-identical.

FIG. 20 is a schematic of an example terminal transamination method to build (ENZ)ₙ₁-(L)ₙ₂-(CTA)ₙ₃, where a polymer bearing multiple oxime groups is an example linker. Here, n2=1.

FIG. 21 is a schematic of an example of biotin-streptavidin linkage method to build (ENZ)ₙ₁-(L)ₙ₂-(CTA)ₙ₃. Here, n1=n2=n3=1.

FIG. 22 is a schematic of an example of sortase-catalyzed transpeptidation method to build (ENZ)ₙ₁-(L)ₙ₂-(CTA)ₙ₃. Here, n1=n2=n3=1.

FIG. 23 is a schematic of an example of sortase-catalyzed transpeptidation method to build (ENZ)ₙ₁-(L)ₙ₂-(CTA)ₙ₃; here, n2=1. A 4-arm PEG is an example multi-arm PEG as a linker L.

FIG. 24 is a schematic showing examples of carrier protein incorporation by methods of terminal transamination or crosslinking with a multi-arm PEG. The carrier protein is represented by the linker L. Here, the “n” displayed may be non-identical.

FIG. 25 is a schematic showing rapid assembly of building blocks at a 1,3,5-triazine core by successive nucleophilic substitution of cyanuric chloride.

FIG. 26 is a schematic showing an example of building block assembly at the 1,3,5-triazine core.

FIG. 27 is a schematic showing branched indoxyl glucosides with a phenolic hydroxy group present for further functionalization.

FIG. 28 is a schematic showing an example for Formula I, design 1. SEN formation is achieved before ENZ-CTA joining. Here, the “n” displayed may be non-identical.

FIG. 29 is a schematic showing an example for Formula I, design 1. SEN formation is achieved after the ENZ-CTA joining.

FIG. 30 is a schematic showing an example for Formula I, design 2. Here, the “n” displayed may be non-identical.

FIG. 31 is a schematic showing an example for Formula I, design 3. BSA, the carrier protein, is conjugated with multiple CTA, X-PG and ENZ units.

FIG. 32 is a schematic showing an example for Formula II, design 1. Here, the “n” displayed may be non-identical.

FIG. 33 is a schematic showing an example for Formula II, design 2. Here, the “n” displayed may be non-identical.

FIG. 34 is a schematic illustration showing an example for radiosensitizer incorporation in a third agent and example entities upon enzymatic cleavage of the third agent according to some embodiments of the present invention. “R*” indicates a radiolabel. “RS” indicates a radiosensitizer. “WSG” indicates a water-soluble group. “X” indicates a site for enzymatic cleavage. “Z” indicates a modified non-polar molecular entity left after release of the WSG.

### DETAILED DESCRIPTION OF EXAMPLE EMBODIMENTS

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety. In case of a conflict in terminology, the present specification is controlling.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (DCPA 1976) (emphasis in the original); see also MPEP § 2111.03. Thus, the term “consisting essentially of” as used herein should not be interpreted as equivalent to “comprising.”

It will also be understood that, as used herein, the terms “example,” “exemplary,” and grammatical variations thereof are intended to refer to non-limiting examples and/or variant embodiments discussed herein, and are not intended to indicate preference for one or more embodiments discussed herein compared to one or more other embodiments.

The term “about,” as used herein when referring to a measurable value such as an amount or concentration and the like, is meant to encompass variations of ±10%, 5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measureable value may include any other range and/or individual value therein.

“Pharmaceutically acceptable” as used herein means that the compound, anion, or composition is suitable for administration to a subject to achieve the treatments described herein, without unduly deleterious side effects in light of the severity of the disease and necessity of the treatment.

As used herein, the terms “increase,” “increases,” “increased,” “increasing,” “improve,” “enhance,” and similar terms indicate an elevation in the specified parameter of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more.

As used herein, the terms “reduce,” “reduces,” “reduced,” “reduction,” “inhibit,” and similar terms refer to a decrease in the specified parameter of at least about 5%, 10%, 15%, 20%, 25%, 30%, 35%, 40%, 45%, 50%, 55%, 60%, 65%, 70%, 75%, 80%, 85%, 90%, 95%, 97%, or 100%.

Provided according to embodiments of the present invention are compounds and methods useful in brachytherapy (e.g., molecular brachytherapy). A method of the present invention may selectively create and/or provide a deposit (e.g., an immobilized deposit) of radiolabeled compound (i.e., the third agent and/or derivative thereof) in and/or adjacent to a tumor and/or cancer cell, and may leave normal tissue relatively untouched and/or substantially free from the effects of a radiolabeled compound. “Substantially free” as used herein in reference to the effects of a radiolabeled compound means that cell viability of normal tissue and/or normal cells (e.g., non-cancerous cells) from systemic exposure of the radiolabeled compound is not reduced by more than 5%, 10%, 15%, or 20% compared to cell viability of the normal tissue and/or normal cells in the absence of the radiolabeled compound. As those of skill in the art will recognize, due to the bystander effect, there may be some normal tissue(s) and/or normal cell(s) that are damaged and/or are killed due to the effects of the radiolabeled compound. However, according to embodiments of the present invention, systemic exposure of the radiolabeled compound may be minimized such that only normal tissues and/or normal cells in proximity to cancer cells are affected due to the bystander effect and/or certain tissues and/or cells exposed to the radiolabeled compound due to its presence in circulation (e.g., liver and/or kidney cells) may be adversely affected, damaged and/or killed by the radiolabeled compound. Thus, a method of the present invention may provide a localized and/or targeted delivery of a radiolabeled compound, which may minimize adverse effects (e.g., cell death) on normal cells, particularly those outside the range for the bystander effect. In some embodiments, a method of the present invention provides less adverse effects and/or side effects (e.g., reduced cell damage to normal tissue, reduced normal cell death, reduced pain, reduced bleeding, etc.) than a conventional brachytherapy method. In some embodiments, a method of the present invention comprises administering three agents: a first agent, a second agent, and a third agent.

Compounds of the present invention include a first agent (A). The first agent may comprise a cancer cell targeting agent (CTA) such as, e.g., transferrin; a protecting group (PG); a cross-linking moiety (X); and an enzyme (ENZ). All or a portion (e.g., the enzyme, protecting group, and/or cross-linking moiety of the first agent) of the first agent can be and/or is configured to be internalized into a cell (e.g., a cancer cell). At least a portion of the first agent including the enzyme and one or more protecting groups and/or one or more degradation shielding moieties can be internalized into a cell (e.g., a cancer cell). In some embodiments, a protecting group of the first agent is an enzymatically cleavable protecting group, which may be cleaved in vivo, such as, e.g., in the endosome and/or lysosome of a cancer cell. Upon cleavage of the protecting group, an exposed cross-linking moiety may engage in cross-linking in situ, thereby affording and/or providing a matrix containing the enzyme of the first agent (matrix-ENZ). “Matrix-ENZ” is also referred to herein as a derivative of the first agent. Cleavage of the protecting group by one or more enzyme(s) present in a cell (e.g., in the lysosome) can cause immobilization of the matrix-ENZ inside the cell (e.g., a cancer cell), thereby providing an immobilized matrix. “Immobilized matrix” as used herein in reference to matrix-ENZ refers to a composition comprising the enzyme of the first agent with one or more of the cross-linking moieties of the first agent cross-linked, and the composition being in a form and/or size that does not allow for a majority of the composition to move outside of the cell in which it is present when a second agent is not in contact with the cell and/or administered to a subject. Further, when the second agent is and/or was in contact with the cell and/or administered to the subject, “immobilized matrix” refers to the composition being in a form and/or size that does not allow for a majority of the composition to move from the extracellular space. Thus, an immobilized matrix in the absence of a second agent means that the enzyme (i.e., the enzyme of the first agent) present in the composition is not freely diffusible into the drainage of the fluid in the extracellular space (ECS) and/or the immobilized matrix renders the enzyme substantially or completely immobile and/or retained inside the cell. However, in the presence of a second agent, an immobilized matrix that has been released from the cell is not freely diffusible from the extracellular space and/or is substantially or completely immobile and/or retained in the extracellular space. “Substantially” as used herein in reference to a compound or agent (e.g., matrix-ENZ) being immobile or retained in a given location (e.g., inside a cell) means that less than 10% (e.g., less than about 5%, 1%, 0.5%, or 0.01%) of the compound or agent can move to the different stated location (e.g., outside the cell). In some embodiments, an immobilized matrix is insoluble or has a low solubility inside a cell and/or a component thereof and/or is precipitated inside a cell and/or a component thereof. Insolubility or reduced solubility compared to a different form is one way to create immobility.

Depending on the nature of the cancer cell targeting agent, some amount of matrix-ENZ may be formed in normal cells (i.e., non-cancerous cells); however, a greater amount of the matrix-ENZ may be formed in cancer cells compared to normal cells. In some embodiments, matrix-ENZ is formed in cancer cells in an amount that is 2, 3, 4, 5, 6, 7, 8, 9, 10, 20, 30, 40, or 50-fold greater or more than the amount formed in normal cells. The matrix-ENZ may have protection against endogenous degradative enzymes (e.g., proteases, glycosidases, disulfide reductases, etc.) in the cell in which it is present, particularly in the lysosome. In some embodiments, the enzyme in the matrix-ENZ may have activity toward one or more (e.g., 1, 2, 3, 4, or more) substrates such as, e.g., one or more non-native substrates.

Any suitable cancer cell targeting agent may be used in the present invention. In some embodiments, the first agent comprises a cancer cell targeting agent that binds to and/or targets an endocytosing receptor or other internalizing unit on a cell (e.g., a cancer cell), and the endocytosing receptor or other internalizing unit may be overexpressed in cancer cells relative to normal cells. In some embodiments, the endocytosing receptor, other internalizing unit, and/or other target of the cancer cell targeting agent may be unique to cancer cells. In some embodiments, the cancer cell targeting agent is any agent or compound that directs the first agent to a given or target cellular destination. In some embodiments, the cancer cell targeting agent directs the first agent from outside a cell (e.g., a cancer cell) across and through the plasma membrane of the cell, into the cytoplasm of the cell, and optionally into a cell organelle (e.g., the lysosome of the cell). Example cancer cell targeting agents include, but are not limited to, polypeptides such as antibodies; viral proteins such as human immunodeficiency virus (HIV) 1 TAT protein or VP22; cell surface ligands; peptides such as peptide hormones; and/or small molecules such as hormones or folic acid. Further example cancer cell targeting agents include, but are not limited to, those described in U.S. Pat. No. 7,807,136 and 7,615,221. In some embodiments, the endocytosing receptor or other internalizing unit for the cancer cell targeting agent is expressed on cancer cells at a concentration that is greater than non-cancerous cells such as, for example, at a concentration that is about 2, 3, 4, 5, 6, 7, 8, 9, or 10-fold higher or more.

In some embodiments, the enzyme of the first agent and matrix-ENZ may be a protein, ribozyme, abzyme, or abiological catalyst. The enzyme may have activity toward a substrate that is not native in a cell (e.g., a cancer cell). In some embodiments, the enzyme lacks activity toward native substrates in a cell (e.g., a cancer cell) and/or the enzyme is heterologous to a subject that the enzyme/first agent is administered to. The enzyme may be largely (e.g., greater than 50%, 60%, 70%, 80%, 90%, or 95%) if not entirely (i.e., 100%) heterologous to native enzymes present in a subject to which the enzyme/first agent is administered. The origin of the heterologous enzyme may be derived from Archaea or bacteria, prepared by directed evolution from an existing (e.g., mammalian, Archaean, or bacterial) enzyme, and/or constructed de novo upon computational or intuitive considerations. In some embodiments, the enzyme may exhibit a high activity toward one or more substrates of a type that is not found in the subject or of a type that is not found in substantial quantities in organs to which the third agent is substantially exposed (e.g., kidney, liver).

The enzyme may be a native biological product that is modified to bear only a single linker to connect to the cancer targeting agent, or can be derivatized in various ways. The derivatization can include, but is not limited to, attachment of PEG groups (i.e., PEGylation) or other groups to alter solubility and/or circulation time of the enzyme, attachment of a linker bearing a cross-linkable group that itself is protected (L-X-PG), compartmentalized in a superstructure (e.g., a single enzyme nanogel (SEN)), and combinations of the aforementioned derivatization methods. An example of the latter entails a SEN that bears a collection of PEG groups, a collection of L-X-PG groups, and a single linker for attachment to the cancer targeting agent. In some embodiments, the enzyme is a single enzyme nanogel. A “single enzyme nanogel” or “SEN” as used herein refers to an enzyme that has been derivatized with one or more cross-linking agents to form a protective layer about the enzyme. In some embodiments, an SEN is an enzyme encased in an oligomeric or polymeric outer layer that affords resistance to degradation of the enzyme. An example of an SEN is one or more polyacrylamide moieties surrounding and/or encapsulating the enzyme. In some embodiments, an SEN is a nanobiocatalyst that includes an enzyme surrounded by and/or embedded in a hydrophilic, polymeric crosslinked nanostructure, such as, e.g., a polyacrylamide nanostructure. In some embodiments, the enzyme is a beta-lactamase, for example, a beta-lactamase that is modified (i.e., a modified beta-lactamase) compared to a native beta-lactamase in a subject. In some embodiments, the enzyme is a phosphatase such as, for example, a thiophosphatase such as, e.g., a thiophosphatase with activity toward cleavage of a thio-substituted phosphate unit, phosphoamidase, or thiophosphoamidase. In some embodiments, the enzyme is a native phosphoamidase that may be present in a subject, but the native phosphoamidase may be modified (e.g., derivatized with one or more moieties and/or functional groups). In some embodiments, the phosphatase may cleave a thiophospho ester and/or may cleave a thiophosphoramidate.

The enzyme of the first agent and matrix-ENZ may be resistant to proteases such as, e.g., resistant to proteases present in a cancer cell, and/or may be resistant to nucleases such as, e.g., resistant to nucleases present in a cancer cell. In some embodiments, the enzyme is resistant to proteases and/or nucleases present in a lysosome of a cell (e.g., a cancer cell).

The enzyme of the first agent and matrix-ENZ may comprise one or more (e.g., 1, 2, 3, 4, 5, 10, 15, or more) degradation shielding moieties. The one or more degradation shielding moieties may be attached to the enzyme such as e.g., directly (such as to a functional group of the enzyme) or via a linker Example linkers that may be used in a first agent include, but are not limited to, linear or branched moieties (e.g., alkyl moieties) and/or carrier proteins. Further example linkers are shown in Scheme 1.

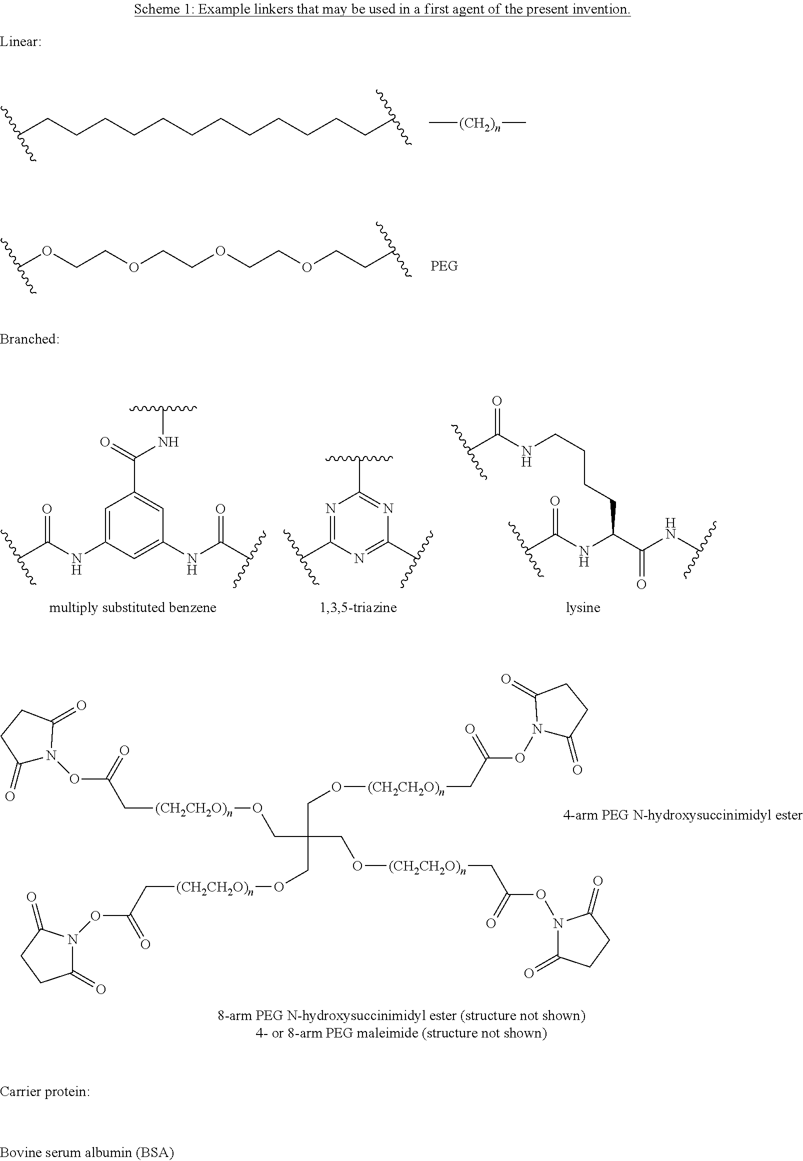

In some embodiments, the one or more degradation shielding moieties protect the enzyme from enzymatic degradation (e.g., proteases and/or disulfide reductases, nuclease degradation, and/or deglycosylation). Example degradation shielding moieties include, but are not limited to, oligoethylene glycol groups and/or polyethylene glycol (PEG) groups. In some embodiments, the one or more degradation shielding moieties are attached to the enzyme via a lysine of the enzyme.

The first agent may comprise one or more (e.g., 1, 2, 3, 4, 5, 6, 7, 8, or more) protecting groups. In some embodiments, a protecting group protects the enzyme from enzymatic degradation (e.g., proteases and/or disulfide reductases, nuclease degradation, and/or deglycosylation). A protecting group may be directly attached to the enzyme (such as to a functional group of the enzyme) and/or a protecting group may be attached to the enzyme via a linker Example linkers include, but are not limited to, a hydrocarbon moiety, a peptoid moiety, an oligoethylene glycol group and/or a polyethylene glycol (PEG) group. In some embodiments, a protecting group is attached to a cross-linking moiety of the first agent, thereby protecting the cross-linking moiety. When the protecting group is removed from the first agent, then the cross-linking moiety may available for cross-linking A protecting group may be configured to cleave and/or may be cleaved from the first agent in vivo in a cell (e.g., a cancer cell), optionally wherein the protecting group cleaves from the first agent in vivo in an endosome and/or lysosome of the cell. A cross-linking moiety of the first agent may and/or may be configured to cross-link in situ in a cell (e.g., a cancer cell). In some embodiments, cross-linking of one or more cross-linking moieties of the first agent precipitates the compound (e.g., matrix-ENZ) in the cell.

The first agent may comprise one or more (e.g., 1, 2, 3, 4, 5, 6, 7, 8, or more) cross-linking moieties. In some embodiments, the first agent comprises at least two protecting groups and at least two cross-linking moieties. In some embodiments, matrix-ENZ is formed upon cross-linking of at least two first agents and/or derivatives thereof and the at least two first agents comprise at least two protecting groups and at least two cross-linking moieties. The number of protecting groups and the number of cross-linking moieties may be the same or different. In some embodiments, the first agent comprises the same number of cross-linking moieties as it does protecting groups, and each protecting group may be attached to a respective cross-linking moiety to protect that cross-linking moiety and/or prevent cross-linking until a given time (e.g., upon cleavage of the protecting group in an endosome and/or lysosome).

The number of protecting groups and/or cross-linking groups may be at least one to 20 or more (e.g., 10, 20, 30, 40, 50, 60, 70, 80, 90, 100 or more). The protecting groups and/or cross-linking group may be designed and/or configured to cause the enzyme to be rapidly and/or irreversibly incorporated into a matrix (e.g., matrix-ENZ). In some embodiments, matrix-ENZ is such that small molecules such as the substrate of the enzyme can gain access to the active site, large molecules such as, e.g., endogenous proteases and other degradative enzymes are precluded, and/or the matrix is immobile or substantially immobile.

The native degradative enzymes of concern include, but are not limited to, proteases, glycosidases, disulfide reductases, and/or nucleases. Resistance of the enzyme to degradation may be built into the structure of the first agent by use of a synthetically tailored enzyme and/or may be created in situ upon cleavage of one or more protecting groups and ensuing formation of the matrix. In some embodiments, the enzyme may be protected from degradation by derivatization with groups such as PEG, derivatization with linker-X-PG groups, the formation of matrix by unveiling and reaction of X groups upon lysosomal cleavage of PG; and/or use of a single enzyme nanogel in the first agent.

In some embodiments, the degradative shield for an enzyme may comprise one or more oligoethylene glycol and/or polyethylene glycol (PEG) group(s), which are known to impart stability and protease resistance to enzymes in vivo. Other known methods to impart degradative resistance include, but are not limited to, the introduction of disulfide linkers to stabilize the enzyme, addition of a blocking group to the N-terminus, and/or the like, which are known methods in the field of enzyme engineering.

In some embodiments, a protecting group of the first agent may be a group that is cleaved with one or more endogenous enzymes in the endosome and/or lysosome of a cell. Example groups that may be cleaved include, but are not limited to, amide groups, phosphoester groups, glycosyl groups, groups that are labile to peroxidases, and/or groups that are known as self-immolative linkers. Such groups can conveniently be attached using standard techniques of bioconjugation to lysine moieties on the enzyme and/or to a carrier protein, linkers and/or the cancer targeting agent. The linker-X-PG groups without cross-linking alone may provide a degradative shield for the enzyme. Removal of the protecting groups (PG) by native enzymatic action can reveal one or more cross-linking moieties, which may undergo self-reaction to create the matrix to which the enzyme is attached and/or present in.

According to embodiments of the present invention, a cross-linking moiety may be unprotected and/or unveiled upon reaching a tumor cell and undergoing intracellular processing. The resulting matrix-ENZ may provide resistance to degradation of the enzyme by native cellular and/or sub-cellular enzymes. The matrix-ENZ that may be formed upon native-enzyme cleavage of the protecting groups (PG) may be largely linear and/or 3-dimensional depending on the number of X-PG groups in each first agent. In some embodiments, the matrix-ENZ may further shield the enzyme from endogenous enzymatic degradation, and in this manner the matrix itself contributes to the degradative shield.

In some embodiments, the enzyme is a single enzyme nanogel (SEN). In some embodiments, use of the SEN may increase thermal stability of the first agent and/or enzyme (e.g., due to suppression of unfolding motions) and/or may retain an increased and/or enhanced level of enzymatic activity compared to the thermal stability and/or enzymatic activity of the enzyme in the absence of the nanogel.

In some embodiments, the first agent has a structure represented by Formula I (with the arrows indicating optional sites of cleavage such as, e.g., by intracellular enzymes):

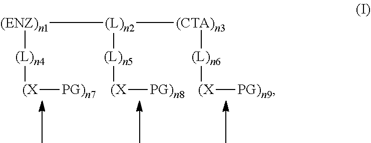

wherein CTA is the cancer cell targeting agent;
L are each an independently selected linking moiety;
PG are each an independently selected protecting group;
X are each an independently selected cross-linking moiety;
ENZ is the enzyme;
n1 and n3 are each independently an integer of 1 or 2 to 10, 50, or 100; and
n2, n4, n5, n6, n7, n8 and n9 are each independently an integer of 0 or 1 to 10, 50, or 100;
wherein of the sum of n7, n8 and n9 is an integer of at least 1.
When n2, n4, n5, n6, n7, n8 or n9 in the compound of Formula I is an integer of 0, then the respective component for that particular integer (e.g., L for n4) is absent. Thus, when n4 is 0, then the respective L (linker) is absent. In some embodiments, the sum of n7, n8 and n9 is an integer of at least 2, 5, 10, 20, 30, 40, 50, 60, 70, 80, 90, or 100.

In some embodiments, the first agent has a structure represented by Formula II (with the arrows indicating optional sites of cleavage such as, e.g., by intracellular enzymes):

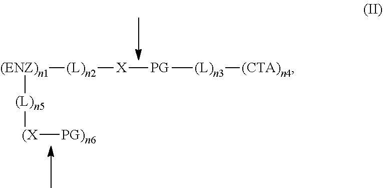

wherein CTA is the cancer cell targeting agent;
L are each an independently selected linking moiety that may be present or absent in the compound;
PG is the protecting group;
X is the cross-linking moiety;
ENZ is the enzyme; and
n1, n4, and n6 are each independently an integer of 1 or 2 to 10, 50, or 100; and
n2, n3, and n5 are each independently an integer of 0, 1, or 2 to 10, 50, or 100.
When n2, n3, or n5 in the compound of Formula I is an integer of 0, then the respective component for that particular integer (e.g., L for n2) is absent. Thus, when n2 is 0, then the respective L (linker) is absent. In some embodiments, n6 is an integer of at least 1, 2, 5, 10, 20, 30, 40, 50, 60, 70, 80, 90, or 100.

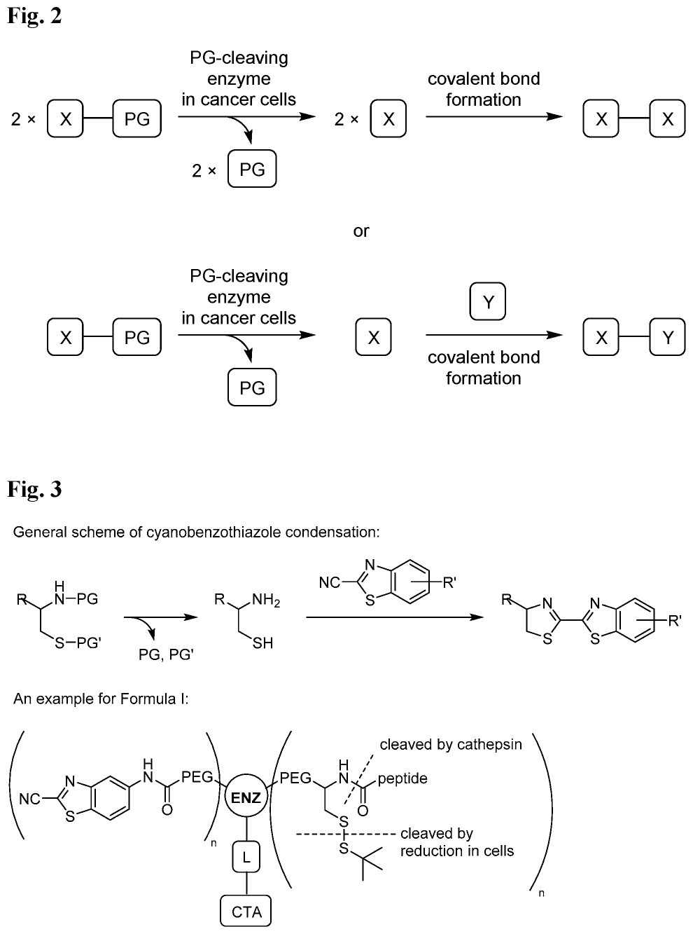

As shown in FIG. 2, each of the cross-linking moieties (X) in Formula I or II can be different (i.e., non-identical). Hetero-crosslinking (X-Y) may occur within non-identical crosslinking groups (X or Y) after PG-cleavage. Referring now to FIG. 3, one type of hetero-crosslinking is illustrated with the top scheme showing cyanobenzothiazole condensation and the bottom scheme showing an example cyanobenzothiazole construct designed for Formula I.

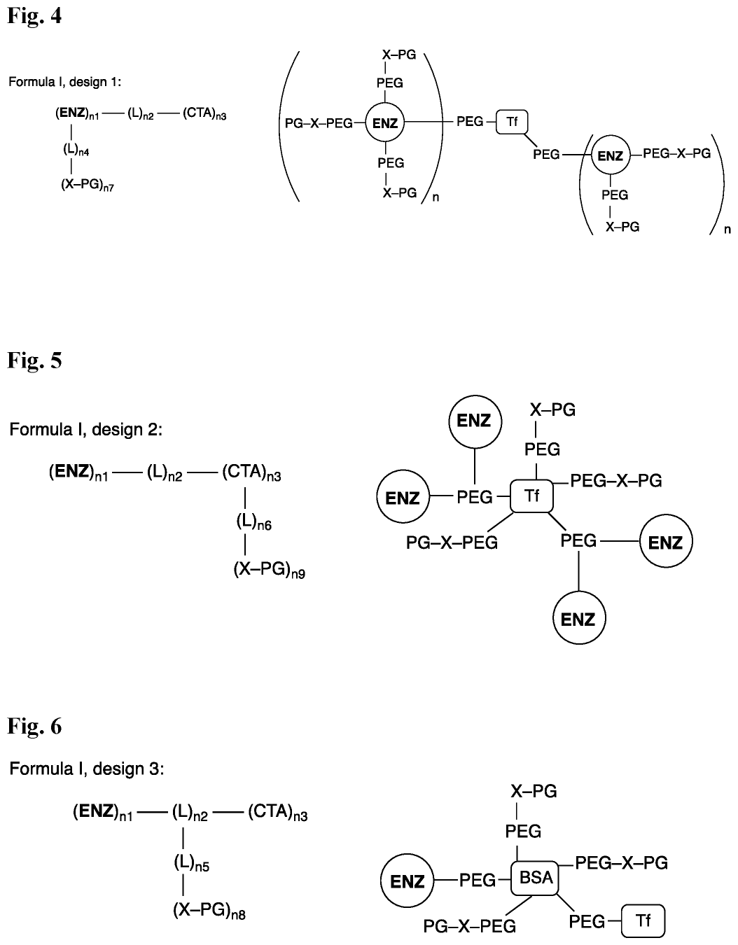

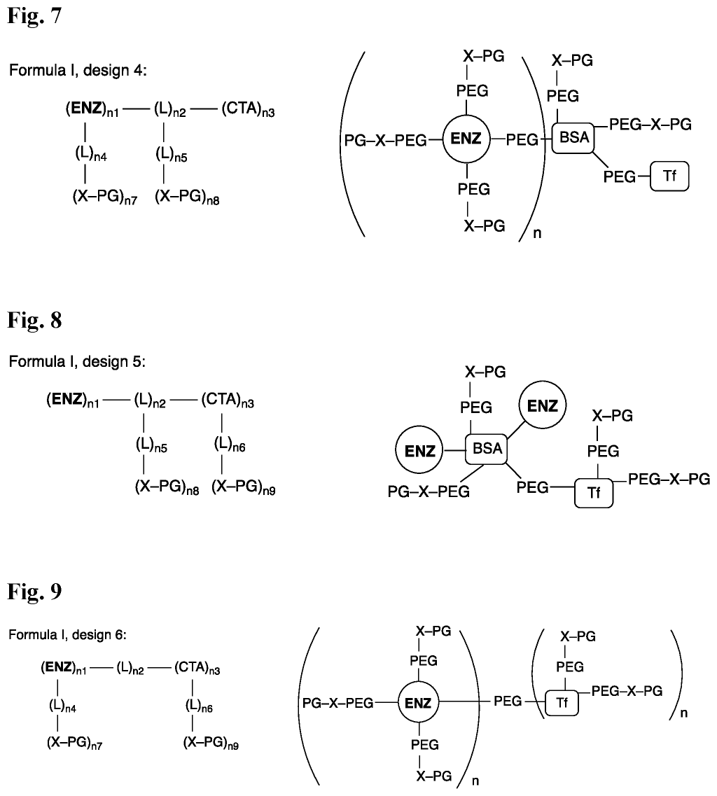

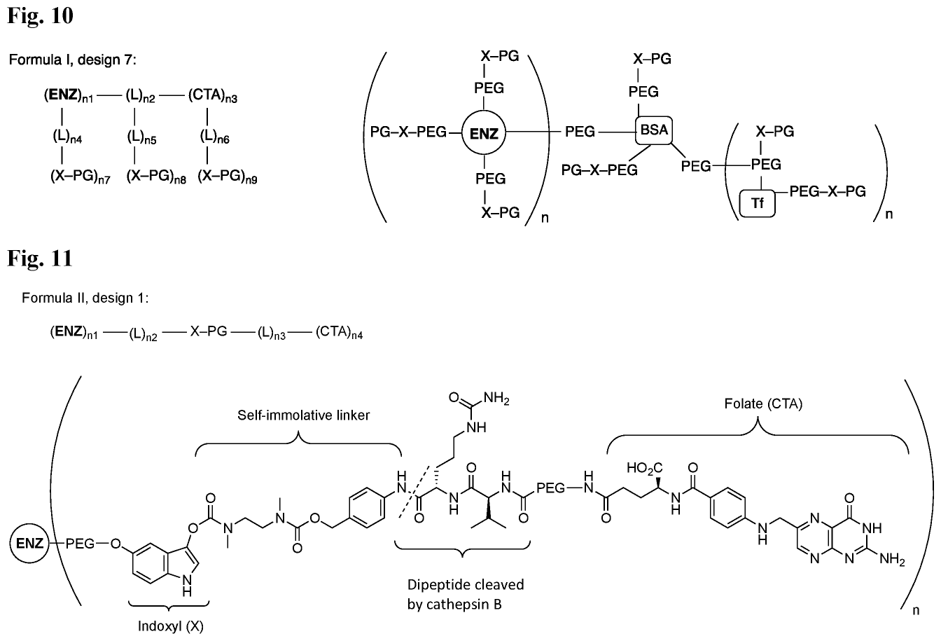

Referring now to FIGS. 4-10, FIGS. 4-10 each show an example generic structure of Formula I and a more specific example first agent of Formula I. FIGS. 4, 5, and 9 each show an example first agent having a structure represented by Formula I in which PEGs are linkers, and transferrin (TO is the CTA. FIGS. 6-8 and 10 each show an example first agent having a structure represented by Formula I in which PEGs and bovine serum albumin (BSA) are example linkers, and transferrin (TO is the CTA. BSA is a carrier protein that may be used as a linker in a first agent of the present invention.

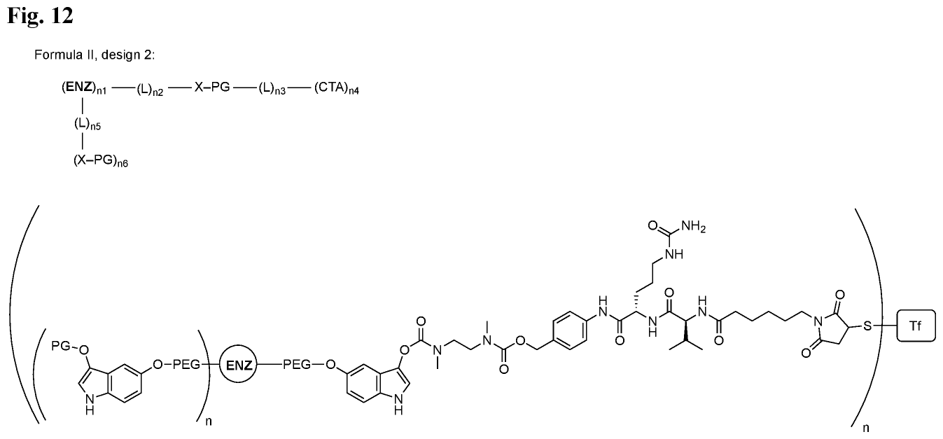

FIGS. 11 and 12 each show an example generic structure of Formula II and a more specific example first agent of Formula II. The example first agent shown in FIG. 11 includes PEGs as linkers, a dipeptide with a self-immolative linker as a protecting group, an indoxyl as a cross-linking moiety, and folate as the CTA. As shown in FIG. 12, the example first agent includes PEGs as linkers, a dipeptide with a self-immolative linker as a protecting group that is between the enzyme and Tf, an indoxyl as a cross-linking moiety, and Tf is the CTA.

The second agent (B) in a method of the present invention may be an agent or compound that can cause cancer cell death (i.e., an anti-cancer agent). In some embodiments, the second agent is a chemotherapeutic agent such as, e.g., an FDA-approved cancer chemotherapeutic agent. Example second agents include, but are not limited to, paclitaxel, taxol, lovastatin, minosine, tamoxifen, gemcitabine, 5-fluorouracil (5-FU), methotrexate (MTX), docetaxel, vincristin, vinblastin, nocodazole, teniposide, etoposide, adriamycin, epothilone, navelbine, camptothecin, daunonibicin, dactinomycin, mitoxantrone, amsacrine, epirubicin and/or idarubicin.

In a method of the present invention only a small amount of the second agent may be administered to a subject, thereby causing lysis of only a fraction of the cancer cells in the subject. Administration of the second agent may cause lysis of about 50% or less of cancer cells in the subject and/or a tumor such as, e.g., about 50%, 45%, 40%, 35%, 30%, 25%, 20%, 15%, 10%, 5%, 1%, or less. In some embodiments, the dose of the second agent in a method of the present invention may be about 0.1% or 1% of that of typical administration in cancer chemotherapy. In traditional cancer chemotherapy, a chemotherapeutic agent is administered in the maximum tolerated dose so as to kill as many cancer cells as possible, and in so doing inadvertently kills many normal cells. The high dose required in traditional chemotherapy is responsible for many adverse effects to normal cells throughout the body. In contrast, a method of the present invention administers a portion of the maximum tolerated dose (e.g., about 1% or less of the maximum tolerated dose) and in doing so may kill primarily cancer cells, optionally killing the supersensitive fraction of cancer cells. Killing of the cancer cells with the second agent can cause lysis of the cells, which then causes the release of matrix-ENZ from the cells (e.g., the supersensitive fraction of cancer cells or most fragile tumor cells) into the tumor extracellular space (ECS).

In some embodiments, the second agent may be administered to a subject in an amount that is about 1%, 0.5%, 0.1%, or less than that of a dose of the second agent that is administered to a subject in the absence of the first agent and/or the third agent. In some embodiments, the second agent may be administered to a subject in an amount that is about 1%, 0.5%, 0.1%, or less than that of the maximum tolerated dose of the second agent.

The third agent (C) in a method of the present invention is a radiolabeled compound that comprises a substrate for the enzyme of the first agent and/or matrix-ENZ. The third agent may convert from a soluble form to a less soluble (e.g., an insoluble) and/or immobile form upon action by the enzyme of the first agent and/or matrix-ENZ. Action by the enzyme of the first agent and/or matrix-ENZ may provide an immobile or substantially immobile deposit of the radioactive portion of the third agent, and the immobile or substantially immobile deposit may remain in the tumor ECS and/or adjacent to one or more cancer cells.

In some embodiments, the enzyme of the first agent and/or matrix-ENZ removes and/or cleaves one or more (e.g., 1, 2, 5, 10, 20, 30 or more) water-solubilizing group(s) (WSG) of the third agent, and removal of the one or more water-solubilizing groups may cause the remaining portion of the third agent including the radioactive entity to aggregate and/or convert to a less soluble (e.g., an insoluble) form. In some embodiments, one or more structural modifications may be made to the WSG of a third agent such that activity by native enzymes is reduced and/or is low or negligible toward the third agent compared to the activity by native enzymes in the absence of the one or more structural modifications. Example water-solubilizing groups include, but are not limited to, a phosphoester (phosphate), thiophosphoester (thiophosphate), dithiophosphoester (dithiophosphate), phosphoamidate, thiophosphoamidate, glycoside, glucuronide, and/or peptide. In some embodiments, a WSG may be directly attached to the third agent such as, e.g., to the portion of the third agent that aggregates upon removal of the WSG group. In some embodiments, a WSG may be attached to the third agent via a linker. The linker may be attached to a portion of the third agent that aggregates upon removal of the WSG (e.g., the radiolabeled aggregation entity) and to the WSG. In some embodiments, the third agent cannot and/or is configured to not be able to enter a cell. Example third agents include, but are not limited to, the Step 4 reagents described in U.S. Pat. Nos. 7,807,136 and 7,615,221. In some embodiments, the third agent comprises a moiety that may be acted on and/or cleaved by a beta-lactamase or phosphatase such as, for example, a thiophosphatase such as, e.g., a thiophosphatase with activity toward cleavage of a thio-substituted phosphate unit, phosphoamidase, or thiophosphoamidase. In some embodiments, the third agent may comprise a β-lactam ring. In some embodiments, the third agent may comprise a thiophospho ester and/or a thiophosphoramidate. In some embodiments, the third agent comprises a thiophosphate unit, which may be cleaved by an enzyme such as, e.g., a thiophosphatase. Cleavage of the thiophosphate unit of the third agent may convert the third agent to a less soluble form and/or may precipitate the remaining portion of the third agent.

In some embodiments, action by the enzyme of the first agent and/or matrix-ENZ (e.g., removal of a WSG) may convert the third agent and/or derivative thereof to a less soluble form in water and/or a bodily fluid (e.g., blood, extracellular fluid, intracellular fluid). In some embodiments, action by the enzyme of the first agent and/or matrix-ENZ (e.g., removal of a WSG) may provide and/or result in a decrease in solubility of the third agent and/or derivative thereof in water and/or a bodily fluid by at least about 10, 20, 30, 40, 50, 60, 70, 80, 90, 100, 1,000, 5,000, 10,000, 50,000, 100,000, 500,000, or 1,000,000-fold or more compared to the solubility in water and/or the bodily fluid of the third agent prior to action of the enzyme (e.g., prior to removal of the WSG).

According to some embodiments of the present invention provided is a method of treating a subject having a solid tumor and/or reducing the size of a solid tumor in a subject, the method comprising: administering a first agent of the present invention to the subject; administering a second agent of the present invention to the subject; and administering a third agent of the present invention to the subject, thereby treating the subject having the solid tumor and/or reducing the size of the solid tumor in the subject.

In some embodiments, a method of the present invention comprises treating a subject having a solid tumor and/or reducing the size of a solid tumor in a subject, the method comprising: localizing a first agent of the present invention in a cancer cell in the subject; releasing the enzyme of the first agent (optionally in the form of an immobilized matrix) from the cancer cell into the extracellular fluid (e.g., into the extracellular space of the tumor); administering a third agent of the present invention and radiotherapy to the subject, wherein the third agent is converted by the enzyme from a soluble form to a less soluble form, thereby treating the subject having the solid tumor and/or reducing the size of the solid tumor in the subject.

A method of the present invention may have the advantage that selectivity for cancer cells compared to normal cells may be multiplicative. For example, if there is a 5:1 accumulation of matrix-ENZ inside cancer cells relative to normal cells after administration of the first agent, and if there is a 10:1 ratio of cancer cells versus normal cells killed and broken open after administration of the second agent, then there is a 50:1 specificity of location of matrix-ENZ in the tumor ECS versus normal ECS after administration of the second agent. The matrix-ENZ in the tumor ECS after administration of the second agent is then exposed to the third agent (whereas matrix-ENZ that is inside normal cells after administration of the second agent is not), which results in deposition of a radiolabeled entity in an immobile or substantially immobile form in the tumor ECS. The approach thus affords brachytherapy at the molecular scale given that the radiolabeled entity is deposited throughout the tumor ECS. The resulting overlapping radiation fields are expected to kill at least a majority (e.g., at about 50%, 55%, 60%, 65%, 70%, 75%, 80%, 85%, 90%, 95%, 97%, 98%, or 99%) or all (i.e., 100%) of the cells in a tumor.

The first agent may be administered prior to the second agent and the third agent. In some embodiments, the first agent, second agent, and/or third agent may be administered in succession with the first agent being administered prior to the second agent and/or the third agent. In some embodiments, the first agent, the second agent, and the third agent are administered in succession with the first agent being administered prior to the second agent and the third agent and with the second agent being administered prior to the third agent.

In some embodiments, the first agent may be administered to the subject about 1 day to about 14 days prior to the second agent and/or the third agent such as, e.g., about 1, 2, 3, 4, 5, 6, 7, 8, 9, 10, 11, 12, 13, or 14 days prior to the second agent and/or the third agent. In some embodiments, the second agent may be administered to the subject at the same time as the third agent. In some embodiments, administration of the second agent and/or third agent to the subject may be at a period in time after which any first agent circulating in the subject (i.e., not internalized in a cell) is allowed to clear. In some embodiments, greater than 50% (such as e.g., at least about 70%, 75%, 80%, 85%, 90%, 95%, 97%, 98%, 99%, or 100%) of the first agent and/or a derivative thereof is cleared and/or removed from the circulation in the subject prior to administration of the second agent, third agent, and/or radiotherapy. In some embodiments, at least about 70%, 75%, 80%, 85%, 90%, 95%, 97%, 98%, 99%, or 100% of the first agent and/or a derivative thereof is cleared and/or removed from the circulation in the subject prior to administration of the third agent and/or radiotherapy. In some embodiments, if the first agent is not cleared from the circulation of the subject, then administration of the third agent may result in deposition of radiolabeled substrate throughout the body of the subject, which may result in loss of selectivity of cancerous versus normal tissue.

A method of the present invention may be carried out over about 2 days to about 14 days or about 1, 2, 3, 4, 5, 6, 7, or 8 weeks. In some embodiments, administration of a first agent, second agent, and/or third agent to a subject may be completed within about 2, 3, 4, 5, 6, 7, 8, 9, 10, 11, 12, 13, or 14 days. In some embodiments, one or more steps of a method of the present invention may be repeated (e.g., one or more administrations of a first agent, second agent, and/or third agent), optionally at the same time as one or more steps and/or at a point in time that is different than one or more steps. For example, in some embodiments, the second agent may be administered to a subject on the same day that the third agent is administered to the subject and then another dose of the third agent may be administered to the subject on a subsequent day, optionally without administration of a first agent, second agent, and/or third agent.

Any suitable method of administration may be used to administer the first agent, the second agent, and/or the third agent to the subject. In some embodiments, the first agent, the second agent, and/or the third agent may be administered to the subject intravenously (e.g., as intravenous infusions), optionally as two or three separate intravenous infusions. In some embodiments, the first agent may be administered to a subject separately (e.g., as a separate intravenous infusion) from the second agent and the third agent. In some embodiments, a composition comprising the second agent and the third agent may be administered to the subject. Thus, the method may consist of a 2-step drug administration process (i.e., (1) administration of the first agent which can build matrix-ENZ inside cancer cells, and (2) administration of a composition containing the second agent and the third agent, the third agent including a substrate for the enzyme of the matrix-ENZ). In some embodiments, a method of the present invention may consist of a 3-step drug administration process. In some embodiments, the first agent, the second agent, and/or the third agent may be administered to the subject via bolus or continuous infusion.

It is well known that some cells within a tumor and/or some regions within a tumor may be hypoxic. Without wishing to be bound to any particular theory, the hypoxia is thought to result from rapid cell growth and division as well as restricted blood supply. Low concentration of oxygen may limit the effectiveness of radiotherapy due to the diminished formation of reactive oxygen species (ROS) upon application of radiation. Radiosensitizers may cause formation of reactive species that may mimic the role of ROS. A radiosensitizer may cause hypoxic cells to be more sensitive (i.e., less resistant) to the damaging effects of radiation. A method of the present invention may comprise administering a radiosensitizer to a subject. A “radiosensitizer” as used herein refers to an agent that increases the sensitivity of one or more cancer cell(s) to radiation. Radiosensitizers are known in the art and may include, but are not limited to, amifostine; clofibrate; efaproxiral; pentoxifylline; metronidazole; misonidazole; etanidazole; pimonidazole; nimorazole; sanazole; nitracrine; tirapazamine; RSU1069; RB6145; capecitabine; AQ4N; temozolomide; AG14361; lisofylline; gemcitabine; camptothecin; L788,123; vandetanib; geftinib; buthionine sulfoximine; celecoxib; and analogues thereof In some embodiments, a radiosensitizer may be a member of the nitroimidazole family. Structures of additional example radiosensitizers based on the nitroimidazole family are shown in Scheme 2, and further structures of radiosensitizers are described in Wardman P., “Chemical radiosensitizers for use in radiotherapy,” Clinical Oncology, 2007, 19(6), 397-417, incorporated herein by reference.

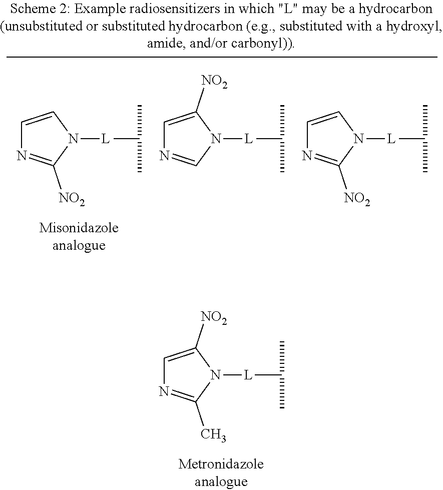

In some embodiments, the first agent, second agent, and/or third agent may be administered in conjunction with a radiosensitizer. A radiosensitizer administered in conjunction with the first agent, second agent, and/or third agent may be separate from the first agent, second agent, and/or third agent (e.g., present as a separate compound and/or in a separate composition administered to the subject) or may be part of the first agent, second agent, and/or third agent. In some embodiments, the third agent may be administered in conjunction with a radiosensitizer. In some embodiments, a radiosensitizer may be administered to a subject in the same composition as the first agent, second agent, and/or third agent, optionally as a separate compound than the first agent, second agent, and/or third agent. In some embodiments, a radiosensitizer may be simultaneously and/or concurrently administered to a subject with a first agent, second agent, and/or third agent such as, e.g., in the same composition or a different composition than the first agent, second agent, and/or third agent.

In some embodiments, a radiosensitizer may be incorporated into (e.g., attached to, linked to, bound to, etc.) a third agent. For example, in some embodiments, a radiosensitizer as shown in Scheme 2 may be covalently attached to a portion of a third agent via L, which may be an unsubstituted or substituted hydrocarbon (e.g., a C1-C20 alkyl, alkenyl, or alkynyl). The radiosensitizer may be released from the third agent upon enzymatic cleavage of an enzyme of a first agent and/or matrix-ENZ. An example design of a radiosensitizer incorporated into a third agent is shown in FIG. 34. In the example third agent shown in FIG. 34, enzymatic cleavage (e.g., by a first agent and/or matrix-ENZ) releases the water-solubilizing group (WSG) attached to the radiosensitizer (RS) and leaves a modified molecular entity (Z) that is not polar. The radiolabeled entity left after enzymatic cleavage may be water-insoluble and/or may be immobilized such as, e.g., via aggregation and/or insolubilization. The radiosensitizer portion may not be immobilized and may diffuse within the tumor space and/or beyond into healthy tissue. While not wishing to be bound to any particular theory, it is believed that the radiosensitizer has effect only in the presence of the radiolabel (i.e., in the presence of the radiolabeled deposit), and thus is ineffectual upon systemic diffusion away from the presence of the radiolabel. Thus, in some embodiments, the third agent may be administered with an incorporated (e.g., attached, linked, and/or bound) radiosensitizer. In some embodiments, responsive to administering the first agent to the subject, the first agent may be transported into at least a portion of cancer cells present in the subject. One or more of the protecting group(s) of the first agent may be removed (e.g., enzymatically cleaved) from the first agent in the portion of cancer cells. In some embodiments, the one or more protecting group(s) may be removed in an endosome and/or lysosome of a cancer cell. In response to removing the one or more protecting group(s) from the first agent, the derivative of the first agent (i.e., the portion of the first agent after removal of the protecting group(s) that includes the enzyme) and/or enzyme of the first agent may be immobilized and/or precipitated in the cancer cell. In some embodiments, the derivative of the first agent and/or enzyme may be immobilized and/or precipitated responsive to cross-linking of the one or more cross-linking moieties of the first agent and/or derivative thereof.

According to some embodiments, after administration of a first agent to a subject, the enzyme of the first agent may be localized in a cancer cell in an immobilized matrix (matrix-ENZ), optionally localized in an endosome and/or lysosome of a cancer cell. The immobilized matrix may be a three-dimensional matrix, which may be formed by cross-linking a cross-linking moiety attached to the enzyme. In some embodiments, one or more cross-linking moieties of a first agent are cross-linked with one or more cross-linking moieties of another first agent present in the cell. In some embodiments, one or more cross-linking moieties of a first agent are cross-linked together. In some embodiments, immobilized matrix may protect the enzyme from enzymatic degradation (e.g., proteases and/or disulfide reductases, nuclease degradation, and/or deglycosylation). In some embodiments, the enzyme in the immobilized matrix converts the third agent to a less soluble (e.g., an insoluble) form and/or the enzyme is configured to convert the third agent to a less soluble form. The less soluble form of the third agent or derivative thereof may be immobile or substantially immobile from the location at which it is converted such as, e.g., the extracellular fluid of the subject (e.g., the tumor extracellular space) and/or from the location of micrometastases in the subject.

In some embodiments, after administration of the second agent to the subject, the immobilized matrix may be released from at least a portion of cells (e.g., cancer cells) such as, e.g., from about 1% to about 100% of cells containing the immobilized matrix. In some embodiments, the immobilized matrix may be released from about 1%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of cancer cells containing the immobilized matrix. In some embodiments, the immobilized matrix may be released from less than about 10% non-cancerous cells containing the immobilized matrix such as, e.g., less than about 9%, 8%, 7%, 6%, 5%, 4%, 3%, 2%, 1%, 0.5% or 0.1%. In some embodiments, after administration of the second agent to the subject, the immobilized matrix may be localized in tumor extracellular space in the subject. In some embodiments, at a time after administration of the second agent to the subject and prior to radiotherapy, at least a majority of the immobilized matrix that is present outside the cell of a subject may be present in tumor extracellular space in the subject, such as, e.g., at least about 50%, 55%, 60%, 65%, 70%, 75%, 80%, 85%, 90%, 95%, 96%, 97%, 98%, 99%, or 100% of the immobilized matrix that is outside a cell of a subject may be localized in tumor extracellular space in the subject.

In some embodiments, responsive to administering the second agent to the subject, a first portion of the immobilized matrix and/or enzyme is released into extracellular fluid (e.g., into the extracellular space of a tumor). Releasing the first portion of the immobilized matrix and/or enzyme into extracellular fluid may comprise releasing the immobilized matrix and/or enzyme from a first plurality of cancer cells in the subject, optionally wherein the first plurality of cancer cells comprises a hyper-sensitive fraction of cancer cells (e.g., cancer cells that are hyper-sensitive to the second agent (e.g., a chemo-sensitive fraction of cancer cells)). In some embodiments, releasing the first portion of the enzyme into extracellular fluid comprises lysing and/or killing the first plurality of cancer cells in the subject.

The third agent may be converted from a soluble form to a less soluble form by the enzyme present in the immobilized matrix. Imaging using methods known to those of skill in the art such as, for example, nuclear medicine imaging techniques (e.g., single photon emission computed tomography (SPECT) and/or positron emission tomography (PET)), computed tomography, and/or magnetic resonance imaging (MRI), may be used to determine the location of the third agent (optionally in insoluble form) in the subject. In some embodiments, imaging of the subject is performed after administration of the third agent to the subject such as, e.g., within about 1, 2, 3, 4, 5, 6, 7, or 8 hours after administration of the third agent to the subject and/or after about 1, 2, 3, 4, 5, 6, 7, 8, 9, 10, 11, 12, 13, or 14 days or weeks after administration of the third agent to the subject.

A method of the present invention may comprise generating radiation fields that span a plurality of cancer cells in a subject, and the radiation fields may minimally contact and/or reach to normal cells (e.g., less than about 20%). In some embodiments, a method of the present invention may comprise generating overlapping radiation fields in the subject. The overlapping radiation fields may be localized in the area of the cancer cells and/or solid tumor in the subject. In some embodiments, responsive to administering the third agent and/or radiation therapy to the subject, a second plurality of cancer cells in the subject are lysed and/or killed, which may release a second portion of the immobilized matrix and/or enzyme into extracellular fluid (e.g., into the extracellular space of the tumor) of the subject. “Radiation therapy” and “radiotherapy” are used interchangeably herein and refer to accumulation of a third agent and/or derivative thereof (e.g., immobilized and/or aggregated third agent and/or a derivative thereof) in an amount sufficient to kill and/or lyse a cell (e.g., cancer cell) in a subject. Accumulation of a sufficient amount of the third agent and/or derivative thereof can provide localized radiotherapy in a subject (e.g., at the location of a tumor and/or micrometastasis in the subject). In some embodiments, a low dose of the third agent may be administered to a subject, which may reduce systemic exposure and/or side effects to the subject, and a method of the present invention may provide for the localized accumulation of the third agent and/or derivative thereof in the vicinity of cancer cells (e.g., in tumor extracellular space and/or at micrometastases). Release of the second portion of the immobilized matrix and/or enzyme may occur due to radiation therapy, which may result in one or more additional releases of the immobilized matrix and/or enzyme into extracellular fluid (e.g., into the extracellular space of the tumor) of the subject. In this manner, a method of the present invention may provide increasing concentrations of immobilized matrix and/or third agent and/or a derivative thereof in the extracellular fluid of the subject and/or in the location of micrometastases.

In some embodiments, the concentration of the enzyme in the extracellular fluid (e.g., tumor extracellular space) of the subject increases as the third agent is converted from a soluble form to a less soluble (e.g., an insoluble) form by the enzyme and/or as radiotherapy is administered. As stated above, the second agent may cause lysis of the supersensitive fraction of cells in the tumor, thereby releasing the matrix-ENZ into the extracellular space of the tumor. There, as the solubility of the third agent is reduced (e.g., converts from soluble-to-less soluble form), the ensuing buildup of radioactive material in the extracellular space damages adjacent, nearby, and/or distant cells (depending on α- or β-decay as is well known, referred to as the bystander effect). The damaging effects of the radioactive decay and the low dose of second agent continue (additively or synergistically) to cause lysis of labile cells (e.g., cancer cells), releasing additional matrix-ENZ. The resulting effect may be autocatalytic and/or amplifying with regard to the quantity of radiolabeled material that is concentrated and immobilized in the tumor rather than normal tissue.

According to some embodiments provided are compositions such as, e.g., pharmaceutical compositions. A pharmaceutical composition of the present invention may comprise a therapeutically effective amount of a compound of the present invention (e.g., a first agent, a second agent, and/or a third agent as described herein) in a pharmaceutically acceptable carrier. Pharmaceutical carriers suitable for administration of a compound of the present invention include any such carriers known to those skilled in the art to be suitable for the particular mode of administration. In some embodiments, a pharmaceutical composition of the present invention is a composition as described in U.S. Pat. No. 7,807,136 and 7,615,221 with the active ingredient replaced with a compound of the present invention as the active ingredient.

In some embodiments, a compound of the present invention (i.e., active ingredient) may be formulated as the sole pharmaceutically active ingredient in the composition or may be combined with other active ingredients.

A composition of the present invention may comprise one or more compounds of the present invention. In some embodiments, the compounds may be formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In some embodiments, the compounds described herein are formulated into pharmaceutical compositions using techniques and procedures well known in the art (see, e.g., Ansel, Introduction to Pharmaceutical Dosage Forms, Fourth Edition 1985, 126).

In the compositions, effective concentrations of one or more compounds or pharmaceutically acceptable derivatives thereof may be (are) mixed with a suitable pharmaceutical carrier. The compounds may be derivatized as the corresponding salts, esters, enol ethers or esters, acetals, ketals, orthoesters, hemiacetals, hemiketals, acids, bases, solvates, hydrates or prodrugs prior to formulation. The concentrations of the compounds in the compositions may be effective for delivery of an amount, upon administration, that treats cancer and/or one or more of the symptoms in a subject and/or kills one or more cancer cells in a subject.

In some embodiments, the compositions are formulated for single dosage administration. To formulate a composition, the weight fraction of a compound of the present invention is dissolved, suspended, dispersed or otherwise mixed in a selected carrier at an effective concentration such that the treated condition is relieved, prevented, or one or more symptoms may be ameliorated.

The active compound may be included in the pharmaceutically acceptable carrier in an amount sufficient to exert a therapeutically useful effect in the absence of undesirable side effects on the subject treated. The therapeutically effective concentration may be determined empirically by testing the compounds in in vitro and in vivo systems described herein and in U.S. Pat. No. 5,952,366 to Pandey et al. (1999) and then extrapolated therefrom for dosages for humans.

The concentration of active compound in the pharmaceutical composition may depend on absorption, inactivation and excretion rates of the active compound, the physicochemical characteristics of the compound, the dosage schedule, and/or the amount administered as well as other factors known to those of skill in the art. For example, the amount that is delivered may be sufficient to kill one or more cancer cells as described herein.

In some embodiments, a therapeutically effective dosage should produce a serum concentration of the active ingredient of from about 0.1 ng/ml to about 50-100 ug/ml. In one embodiment, a therapeutically effective dosage is from 0.001, 0.01 or 0.1 to 10, 100 or 1000 mg of active compound per kilogram of body weight per day. Pharmaceutical dosage unit forms may be prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN™, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration may be sufficient for ameliorating the symptoms of the disease, disorder or condition treated and may be empirically determined.

The pharmaceutical compositions may be provided for administration to humans and/or animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof The pharmaceutically therapeutically active compounds and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions may, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, in one embodiment 0.1-95%, in another embodiment 75-85%.

In some embodiments, a composition of the present invention may be suitable for oral administration. Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, in one embodiment, capsules or tablets. The tablets, pills, capsules, troches and the like may contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, gellan gum, sodium carboxymethylcellulose, polyethylene glycol 4000 and cellulose acetate phthalate.

The compound, or pharmaceutically acceptable derivative thereof, may be provided in a composition that protects it from the acidic environment of the stomach. For example, the composition may be formulated in an enteric coating that maintains its integrity in the stomach and releases the active compound in the intestine. The composition may also be formulated in combination with an antacid or other such ingredient. When the dosage unit form is a capsule, it may contain, in addition to material of the above type, a liquid carrier such as a fatty oil. In addition, dosage unit forms may contain various other materials which modify the physical form of the dosage unit, for example, coatings of sugar and other enteric agents. The compounds may be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials may also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms. Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, xanthan gum, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation. For a solid dosage form, the solution or suspension, in for example propylene carbonate, vegetable oils or triglycerides, is in one embodiment encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(loweralkyl) acetals of loweralkyl aldehydes such as acetaldehyde diethyl acetal.

Parenteral administration, in one embodiment characterized by injection, either subcutaneously, intramuscularly or intravenously is also contemplated herein. Injectables may be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, xanthan gum, hydroxypropyl methylcellulose and polyvinylpyrrolidone. Emulsifying agents include Polysorbate 80 (TWEEN™ 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound may be adjusted so that an injection provides an effective amount to produce the desired pharmacological effect. The exact dose depends on the age, weight and condition of the subject or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.01% or 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

In some embodiments, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

In some embodiments, a composition comprising the first agent, second agent, and/or third agent may be administered to a subject. In some embodiments, a composition comprising the first agent, second agent, and/or third agent may comprise nanoparticles, liposomes, and the like. In some embodiments, the composition is a solution or suspension comprising the first agent, second agent, and/or third agent. In some embodiments, if a delivery vehicle such as, e.g., a nanoparticle or liposome, is employed, then the cancer targeting agent may not be covalently attached to the enzyme, but rather to the delivery vehicle. In some embodiments, the enzyme of the first agent and its linkers may be embedded in the delivery vehicle (e.g., nanoparticle or lysosome), and the cancer cell targeting agent may on the surface of the delivery vehicle.

In some embodiments, a first agent may be administered to the subject in a composition comprising a nanogel. A “nanogel” as used herein refers to a nanometer-scale hydrogel, which comprises crosslinked networks of hydrophilic polymers. Nanogels are known in the art for use in delivering cargoes of biotherapeutics (e.g., protein-containing entities) to cells and to intracellular locales, where the cargo is released by enzymatic triggering or due to distinct physiological state (pH or redox features). Examples of nanogels include, but are not limited to, those described in “Nanogels for Intracellular Delivery of Biotherapeutics,” Li, D.; van Nostrum, C. F.; Mastrobattista, E.; Vermonden, T.; Hennink, W. E. J. Controlled Rel. 2017, 259, 16-28. In some embodiments, when a nanogel is used to administer a first agent, then the cancer cell targeting agent may be on the outside of the nanogel and/or otherwise accessible to interact with the target, and the enzyme, the protecting group, and cross-linking moiety of the first agent may be inside the nanogel. In some embodiments, when a nanogel is used to administer a first agent, then the cancer cell targeting agent and enzyme are not covalently linked.

The present invention finds use in both veterinary and medical applications. Subjects suitable to be treated with a method of the present invention include, but are not limited to, mammalian subjects. Mammals of the present invention include, but are not limited to, canines, felines, bovines, caprines, equines, ovines, porcines, rodents (e.g. rats and mice), lagomorphs, primates (e.g., simians and humans), non-human primates (e.g., monkeys, baboons, chimpanzees, gorillas), and the like, and mammals in utero. Any mammalian subject in need of being treated according to the present invention is suitable. Mammalian (e.g., human) subjects of both genders and at any stage of development (i.e., neonate, infant, juvenile, adolescent, adult) may be treated according to the present invention. In some embodiments of the present invention, the subject is a mammal and in certain embodiments the subject is a human. Human subjects include both males and females of all ages including fetal, neonatal, infant, juvenile, adolescent, adult, and geriatric subjects as well as pregnant subjects. In particular embodiments of the present invention, the subject is a human adolescent and/or adult. In some embodiments, the subject has or is believed to have cancer, optionally wherein the subject has metastatic cancer.

A method of the present invention may also be carried out on animal subjects, particularly mammalian subjects such as mice, rats, dogs, cats, livestock and horses for veterinary purposes, and/or for drug screening and drug development purposes.

In some embodiments, the subject is “in need of” or “in need thereof” of a method of the present invention, for example, the subject has findings typically associated with cancer and/or a tumor, is suspected to have cancer and/or a tumor, and/or the subject has cancer and/or a tumor.

“Treat,” “treating” or “treatment of” (and grammatical variations thereof) as used herein refer to any type of treatment that imparts a benefit to a subject and may mean that the severity of the subject's condition is reduced, at least partially improved or ameliorated and/or that some alleviation, mitigation or decrease in at least one clinical symptom associated with cancer and/or a tumor is achieved and/or there is a delay in the progression of the symptom. In some embodiments, the severity of a symptom associated with cancer and/or a tumor may be reduced in a subject compared to the severity of the symptom in the absence of a method of the present invention.

In some embodiments, a first agent, second agent, and/or third agent of the present invention may be administered in a treatment effective amount. A “treatment effective” amount as used herein is an amount that is sufficient to treat (as defined herein) a subject. Those skilled in the art will appreciate that the therapeutic effects need not be complete or curative, as long as some benefit is provided to the subject. In some embodiments, a treatment effective amount may be achieved by administering a composition of the present invention. In some embodiments, a second agent may not be administered to a subject in a treatment effective amount.

In some embodiments, a method of the present invention comprises administering a therapeutically effective amount of a first agent, second agent, and/or third agent of the present invention to a subject. As used herein, the term “therapeutically effective amount” refers to an amount of a first agent, second agent, and/or third agent of the present invention that elicits a therapeutically useful response in a subject. Those skilled in the art will appreciate that the therapeutic effects need not be complete or curative, as long as some benefit is provided to the subject.

According to some embodiments, a method of the present invention may provide and/or pre-load an enzyme (e.g., the enzyme of the first agent) in an endosome and/or a lysosome of a plurality of cancer cells (and optionally a portion of non-cancerous cells such as, e.g., less than 10% of non-cancerous cells) and the enzyme may be released from a least a portion of the plurality of cancer cells into the tumor ECS as immobilized matrix (matrix-ENZ). A method of the present invention may provide and/or achieve one or more of the following advantages: (1) the first agent may be modular in nature and may be constructed in a building block fashion using synthetic methods and bioconjugation strategies that are within the state of the art; (2) only a very low dose of the second agent (e.g., chemotherapeutic) may be administered, which may thereby achieve great selectivity in matrix-ENZ release from the cancer cells versus normal cells and in so doing, may mitigate adverse effects of the second agent on normal cells; (3) accumulation of deposited third agent (i.e., the radiolabeled substrate) may be autocatalytic because the small quantity of the second agent causes release of matrix-ENZ to the tumor ECS only from the most fragile cancer cells, yet at the outset of administering the third agent, the ensuing soluble-to-less soluble (e.g., mobile-to-immobile) conversion and deposition of the third agent may cause localized radiotherapy. Such localized radiotherapy may result in and/or cause an increased release of the matrix-ENZ from cancer cells. The more matrix-ENZ that is released, the more enzyme available to cause deposition of the third agent. This process may thus be autocatalytic in the deposition of the third agent in the tumor ECS. Hence, a high degree of selectivity between cancerous and normal tissue may be exercised by a method of the present invention. In some embodiments, upon administration of the second agent, matrix-ENZ may be released from cancer cells in an amount that is 2, 3, 4, 5, 6, 7, 8, 9, 10, 20, 30, 40, or 50-fold greater or more than the amount released from normal cells.

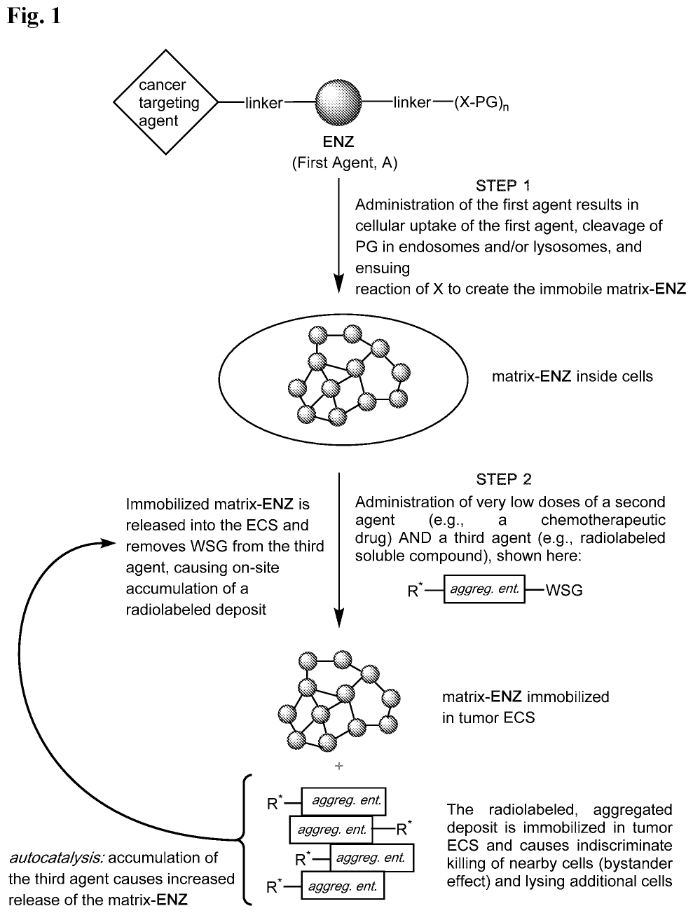

An illustration of an example method is provided in FIG. 1. FIG. 1 shows the autocatalytic process for amplification of the deposition of the third agent by the curved arrow in the schematic. The autocatalytic process is indirect in that the matrix-ENZ promotes concentration and deposition of the third agent, which causes increased bystander cell killing, which releases more matrix-ENZ, and so on. The radionuclide is symbolized by R* and may be any suitable radioisotope such as, e.g., ¹²⁵I or ¹³¹I or other radioisotope, that can be covalently attached to an organic substrate. Covalent attachment as opposed to coordination or chelation affords certainty concerning molecular positioning and is not subject to loss via equilibrium phenomena. In the third agent, the group to be cleaved by action of ENZ is denoted WSG (water-solubilizing group) leaving an entity that may aggregate (i.e., aggregation entity). Removal of the WSG causes deposition of the third agent as a largely immobile material in aggregate or precipitate form. A method of the present invention may immobilize or substantially immobilize the enzyme of the first agent/matrix-ENZ and the third agent in the tumor ECS after administration of the second agent. A method of the present invention may direct and/or incorporate the enzyme of the first agent/matrix-ENZ in and/or to the tumor in a single-step process.

According to some embodiments, a compound (e.g., first agent) and/or method of the present invention may target overexpressed endocytosing receptors of tumor cells. A compound and/or method of the present invention may selectively kill more tumor cells than normal cells by low-dose administration of a second agent (e.g., chemotherapeutic), which may expose matrix-ENZ for conversion and/or immobilization of a radiolabeled substrate. A compound and/or method of the present invention may enzymatically activate the radiolabeled substrate by forming an immobile and/or insoluble deposit in the extracellular space around tumor cells, avoiding unfavorable diffusion over the whole body. Hence, because normal cells have little matrix-ENZ exposed to cause immobilization of the radiolabeled substrate, the method may have a higher selectivity than ADEPT or EMCIT. Both the enzyme (e.g., non-native enzyme) of the first agent and the radiolabeled substrate of the third agent are immobilized in the tumor ECS, and the radiolabeled substrate is accumulated in an autocatalytic manner. The enzymatic process and the autocatalysis for release of the enzyme enables use of a low dose of radiolabeled substrate (e.g., about 50%, 45%, 40%, 35%, 30%, 25%, 20%, 15%, 10% or less of the maximum tolerated dose), which may afford a commensurably low level of adverse effects on normal cells. While the majority of the prodrugs of ADEPT are chemotherapeutic agents, the approach of the present invention employs radiolabeled substrates. In some embodiments, a method of the present invention does not rely on the specificity of antibody-antigen recognition, but may instead rely on the sensitivity of targeted tumor cells against low-dose chemotherapy. Refraining from the limit of tumor-overexpressed antigens opens a wider scope of possible cancer cell targeting agents. In some embodiments, a method of the present invention comprises administering a first agent and the first agent may utilize antibody-antigen recognition for targeting a cancer cell (e.g., the first agent may comprise an antibody for the cancer targeting agent).

Compounds of the present invention can be prepared by methods that are well within the state-of-the-art. In some embodiments, a method for preparation of a first agent of the present invention relies on well-established procedures in the field of bioconjugation chemistry. The purification of reaction mixtures may be achieved by standard methods for separation (e.g., adsorption chromatography, size-exclusion chromatography, ion-exchange chromatography); establishment of purity (e.g., high-performance liquid chromatography); and/or characterization including mass spectrometry (matrix-assisted laser desorption ionization mass spectrometry, electrospray ionization mass spectrometry) and/or nuclear (¹H, ¹³C, and other nuclei) magnetic resonance spectroscopy.

The synthetic approach to preparing a first agent of the present invention may be modular in nature. In some embodiments, the first agent comprises an enzyme (ENZ) and a cancer targeting agent (CTA) joined by a linker, which can include a carrier protein. One or more of the components of the first agent may bear protected cross-linking entities (PG-X) attached via a linker (L). In some embodiments, each component of the first agent may be prepared independently and then joined via standard methods of bioconjugation. The methods can include, but are not limited to, click chemistry processes (alkyne/azide reaction to afford a triazole). The linkers may comprise PEG groups and are typically attached via amidation, which is referred to as PEGylation. Diverse linkers with non-identical reactive end groups (e.g., azide and N-hydroxysuccinimidyl ester) are known and are readily available; such bifunctional linkers include heterotelechelic oligomers and greatly facilitate the synthesis.

The derivatization of the enzyme or CTA may be done in a rational manner (i.e., chiefly affording a single product) or a statistical manner (i.e., inevitably affording a mixture of products). The statistical approach enables a range of the loading of the number of PG-X groups on the ENZ, CTA and/or linker. Regardless of underlying methods employed, the modular construction lends itself to a building block approach, which is amenable to rapid preparation of families of compounds for studies of therapeutic efficacy.

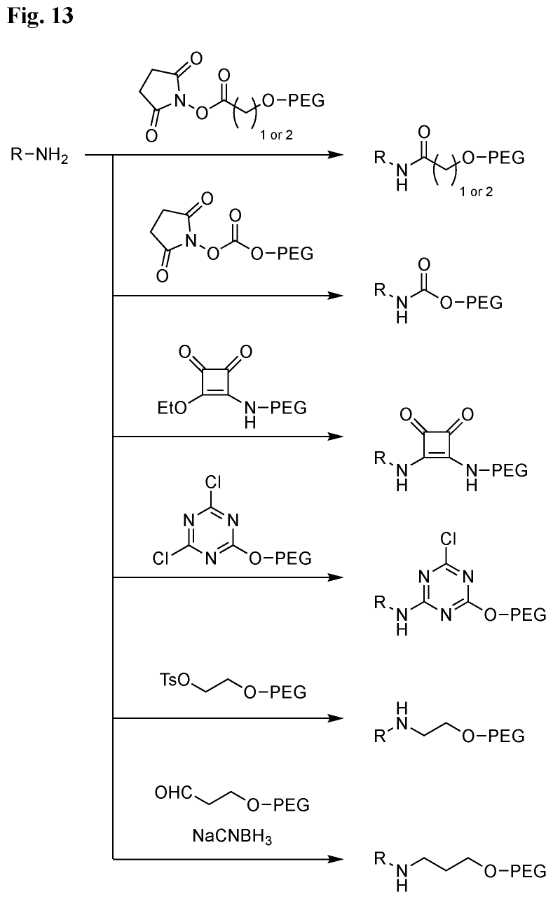

PEGylation of proteins may be carried out by modifying the amino acid residues on the protein surface.^{Vero, Rob,Koni} Among the residues, lysine is a popular target for PEGylation. Several reactive groups have been developed for this purpose and are well known (FIG. 13). The PEG groups can be linear, branched, or dendrimeric.

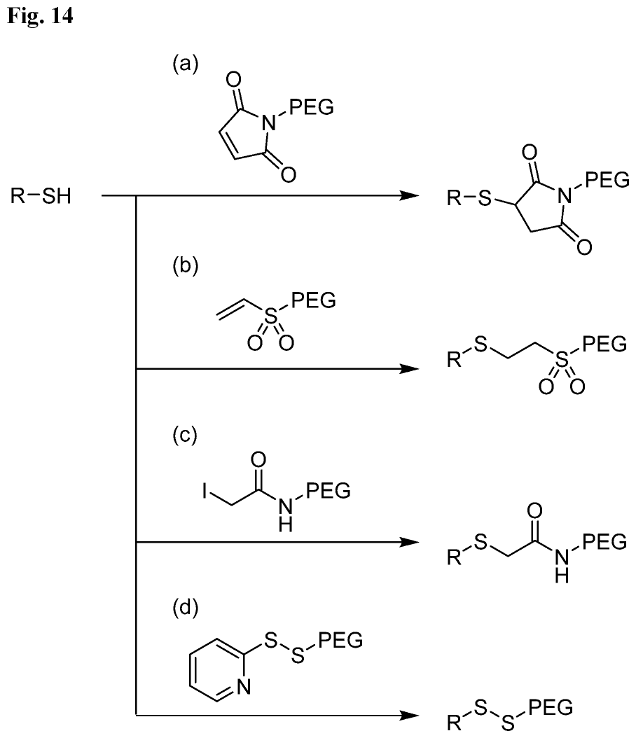

Cysteine is also a useful amino acid residue for PEGylation (FIG. 14). Moreover, the cleavage of cystine disulfides with reducing agents generates additional reactive sulfhydryl groups.

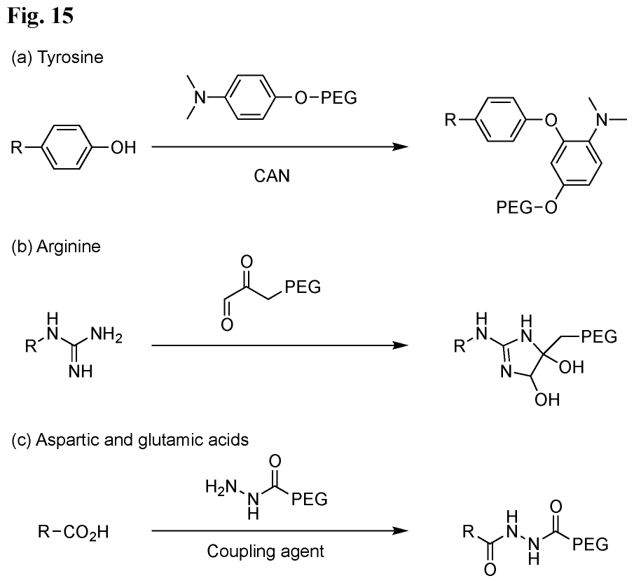

Other amino acid residues such as tyrosine, arginine, and aspartic/glutamic acids can also be targets for PEGylation (FIG. 15).

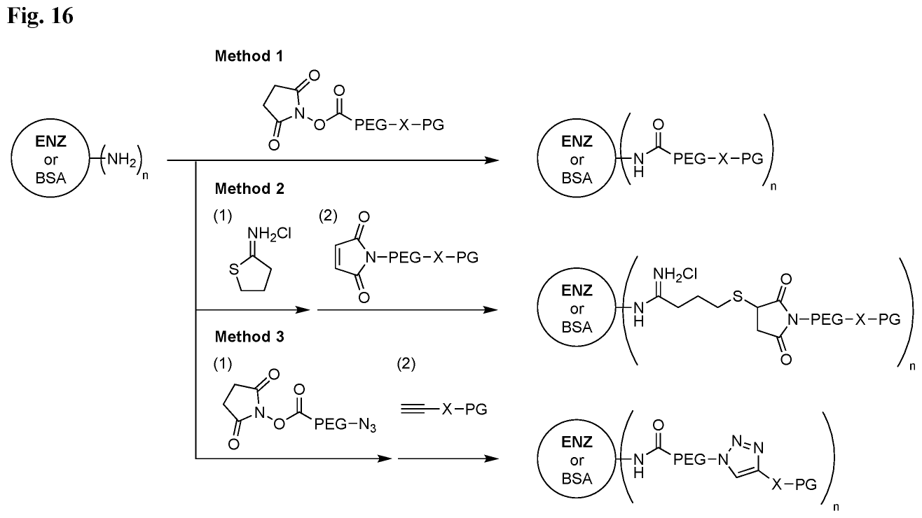

Described below are three general methods to attach a PEG-X-PG group to proteins (FIG. 16). Here, bovine serum albumin (BSA) and lysine are used as examples of a carrier protein and a reactive amino acid, respectively. Method 1 is a 1-step modification of lysine using a PEGylating agent possessing the X-PG group. An N-hydroxysuccinimidyl ester is shown as an example. Method 2 is a functional group conversion followed by the attachment of the PEG-X-PG unit. The thiolation of the lysine and the alkylation with a maleimide-PEG-X-PG is shown as an example. Method 3 entails PEGylation followed by attachment of the X-PG group. Amidation and the alkyne/azide “click” reaction are shown as examples.

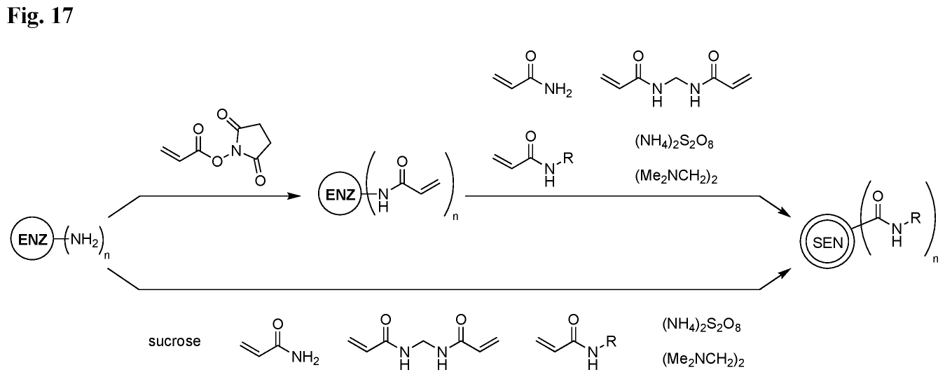

Enzymes encapsulated within a hydrogel (e.g., a thin hydrogel mantle) are known as SENs. Such compartmentalized enzymes may exhibit increased stability under denaturing conditions such as high temperatures and/or solutions with a high content of organic solvent, yet maintain a high level of enzymatic activity.^{Yan} SENs were originally prepared in a two-step procedure (FIG. 17). The first step enatils introduction of vinyl groups attached to lysine residues. Then, in the second step, crosslinking polymerization between the vinyl groups and in the presence of a bis-acrylamide unit forms a polyacrylamide layer on the enzyme surface. Recently, a modified 1-step procedure was reported for SEN preparation. The lysine modification is skipped because of the addition of sucrose during crosslinking polymerization.^{Bedo}

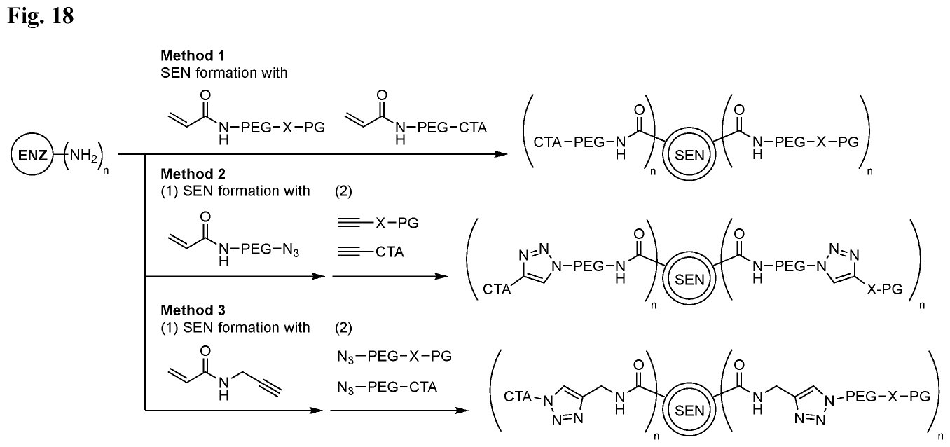

When an N-substituted acrylamide is added during crosslinking polymerization, the resulting SENs have substituents on the surface,^{Gu} typically in a statistical manner. FIG. 18 shows three example methods to introduce both the PEG-X-PG and CTA-PEG groups. In method 1, both groups are attached to an acrylamide unit, which is essential for SEN formation. The X-PG and CTA entities are introduced in the second step in methods 2 and 3. Method 2 uses a PEGylated acrylamide, while a PEG unit is added in the second step in method 3. Alkyne/azide reactions are shown as example click reactions to connect each moiety.

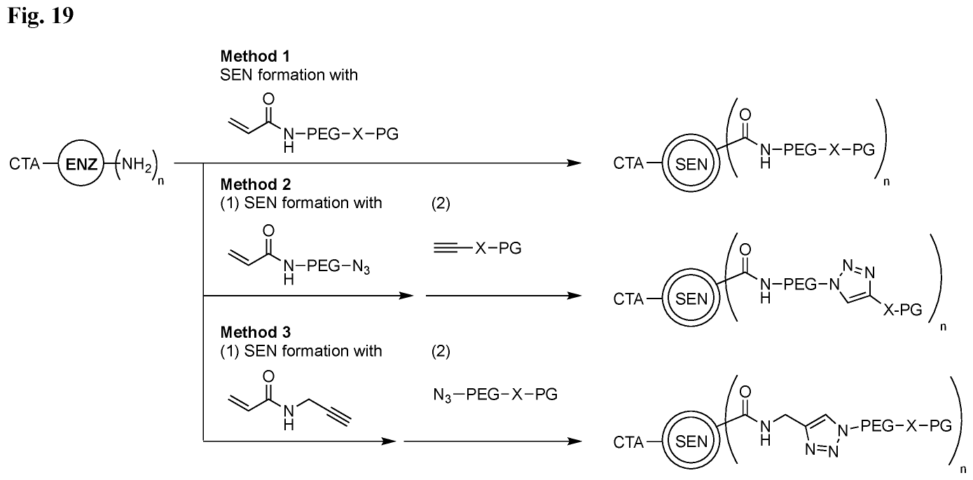

If the ENZ is already connected to a single CTA, the resulting SEN also has a single CTA (FIG. 19). Similar to the general procedures in FIG. 18, there can be three methods to introduce PEG-X-PG groups to SENs. This approach may be best employed for CTA units that are not proteins (e.g., folic acid).

To achieve optimal degradation shielding, it may be desirable to increase the number of X-PG units on the surface of the ENZ in those cases where there are insufficient bio-conjugation sites. On the other hand, some conjugation sites near the active site may impair activity of the ENZ after bioconjugation of the X-PG units. However, to introduce X-PG groups, a CTA unit or a carrier protein to favorable sites on the ENZ, the conjugation sites on the ENZ, mainly lysines, may need to be deactivated. Amine landscaping is a method to control the number and locations of lysines of the ENZ.^{Hown} Briefly, site-directed mutagenesis converts inactive residues to lysines to achieve a high-loading of L or X-PG groups, or avoid the conjugation that impairs activity of the ENZ by converting specific lysines to arginine residues. For the ENZ bearing sufficient lysines in favorable locations, the extent of conjugation may be simply controlled by the linker-to-protein ratio.^{Herm}

In some embodiments, prior to construction of architectures of Formula I, the ENZ and/or CTA may be derivatized with L or X-PG groups via N-hydroxysuccinimidyl ester bioconjugation chemistry with lysine; or maleimide bioconjugation chemistry with cysteine. After installation of the L or X-PG groups, the modified proteins may be applied for joining the ENZ and CTA constituents (as well as any carrier protein), thereby constructing Formula I or II by employing the following methods.

Alternatively, in the case where only a limited number of lysines or cysteines are consumed in the processes for assembly of the ENZ-L-CTA construct, the remaining lysines or cysteines on the surface of proteins may be employed to load the L or X-PG units.

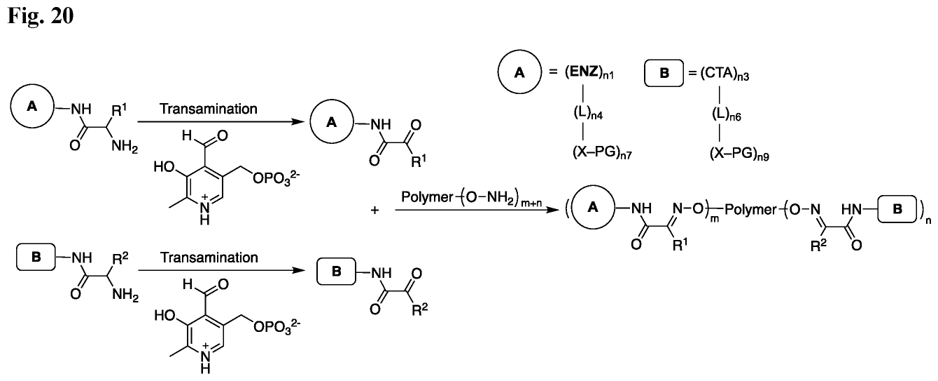

Terminal transamination is a method to selectively modify the N-terminus of peptides and proteins, e.g., myoglobin and eGFP.^{Fran1,Fran2,Fran3,Fran4,Fran5} Application of this known method entails treatment of the ENZ with pyridoxal-5-phosphate under mild conditions (pH 6.5, 37° C., 24 h) to form the terminal oxime, followed by reaction with an alkoxyamine agent at room temperature. This reaction is carried out in aqueous buffer, thereby refraining from denaturing of the protein. A polymer bearing multiple alkoxyamines has been reported to link oxime-terminated proteins.^{Nish1,Nish2,Fran3} With this polymer, the CTA and ENZ are joined to target the cancer cells and traverse the cell membrane via endocytosis. This method will be employed to construct Formula I, design 1, 2 or 6. The polymers bearing an oxime are the linker L in (ENZ)ₙ₁-(L)ₙ₂-(CTA)ₙ₃. Here, n2=1. The loading of L or X-PG on the ENZ or CTA is carried out prior to (ENZ)ₙ₁-(L)ₙ₂-(CTA)ₙ₃ assembly to construct Formula I, design 1, 2 or 6. Alternatively, for Formula I, design 6, loading of L or X-PG can be carried out after (ENZ)ₙ₁-(L)ₙ₂-(CTA)ₙ₃ construction (FIG. 20).

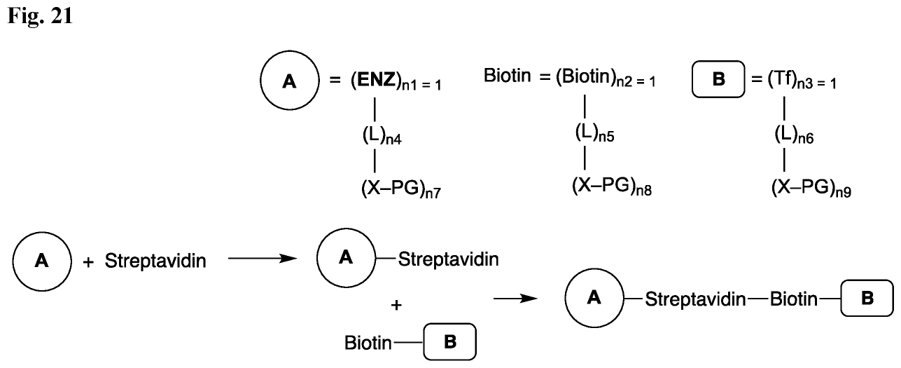

Transferrin has been extensively used for targeting tumor cells in vitro.^{Qin,Faul} Biotinylated transferrin has been successfully used to form a conjugate with streptavidin-linked proteins, for an in vivo study to traverse nasal mucosa and vaginal mucosa of mice.^{Mann} In some embodiments, one approach may be to link streptavidin with the ENZ via bioconjugation, followed by conjugation with commercially available biotinylated transferrin to form (ENZ)ₙ₁-(L)ₙ₂-(Tf)ₙ₃. This method may be employed to construct Formula I, design 1, 2, 5 or 7. Transferrin is the CTA in this method; streptavidin-biotin is the linker L in (ENZ)ₙ₁-(L)ₙ₂-(CTA)ₙ₃; and n1=n2=n3=1. Loading of L or X-PG on the ENZ or CTA is carried out prior to (ENZ)ₙ₁-(L)ₙ₂-(CTA)ₙ₃ assembly to construct Formula I, design 1, 2, 5 or 7. Alternatively, for Formula I, design 7, loading of L or X-PG units can be installed after (ENZ)ₙ₁-(L)ₙ₂-(CTA)ₙ₃ construction (FIG. 21).

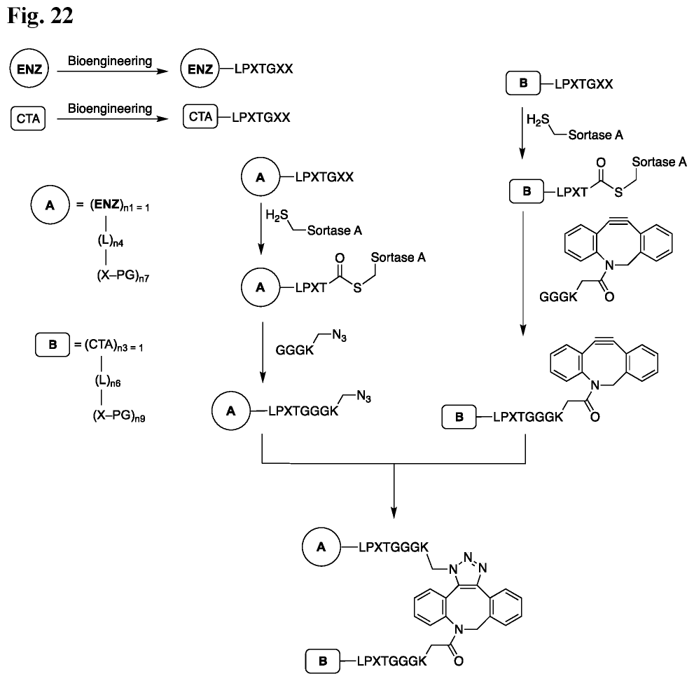

Sortase has been successfully employed to link two proteins in a selective (non-statistical) manner with accompanying azide-alkyne click chemistry.^{Ploe} In some embodiments, to apply this method, a short protein sequence LPXTGXX is linked at the C-terminus of a protein. Cloning techniques can be used to insert the LPXTGXX sequence to the ENZ or CTA (if a protein). This method may be employed to construct Formula I, design 1, 2 or 6. An azide-cyclooctyne unit is the linker L in (ENZ)ₙ₁-(L)ₙ₂-(CTA)ₙ₃; here, n1=n2=n3=1. Loading of the L or X-PG groups on the ENZ or CTA is carried out prior to (ENZ)ₙ₁-(L)ₙ₂-(CTA)ₙ₃ construction, but after the bioengineering of protein—LPXTGXX to construct Formula I, design 1, 2, or 6. Alternatively, for Formula I, design 6, loading of the L or X-PG groups could be achieved after (ENZ)ₙ₁-(L)ₙ₂-(CTA)ₙ₃ construction (FIG. 22).

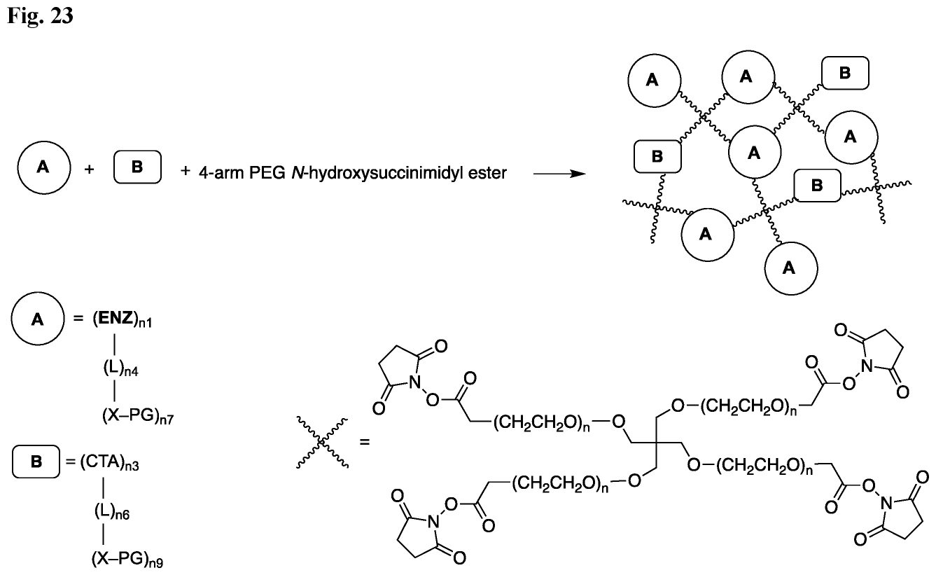

A 2-, 4- or 8-arm PEG N-hydroxy succinimidyl ester or a 2-, 4- or 8-arm PEG maleimide may be used as the linker to conjugate ENZ and CTA at different ratios. Multi-arm PEGS have been reported as successful linkers for branched fusion proteins,^{Pang} or used to build branched polymeric nanoparticles as novel tumor targeting carriers.^{Pras} This architectural feature may be employed to construct Formula I, design 1, 2 or 6. The multi-arm PEG is the linker L in (ENZ)ₙ₁-(L)ₙ₂-(CTA)ₙ₃; n2=1. Loading of L or X-PG units on the ENZ or CTA is carried out prior to (ENZ)ₙ₁-(L)ₙ₂-(CTA)ₙ₃ construction to create Formula I, design 1, 2, or 6. Alternatively, for Formula I, design 6, loading of the L or X-PG units can be carried out after (ENZ)ₙ₁-(L)ₙ₂-(CTA)ₙ₃ construction (FIG. 23).

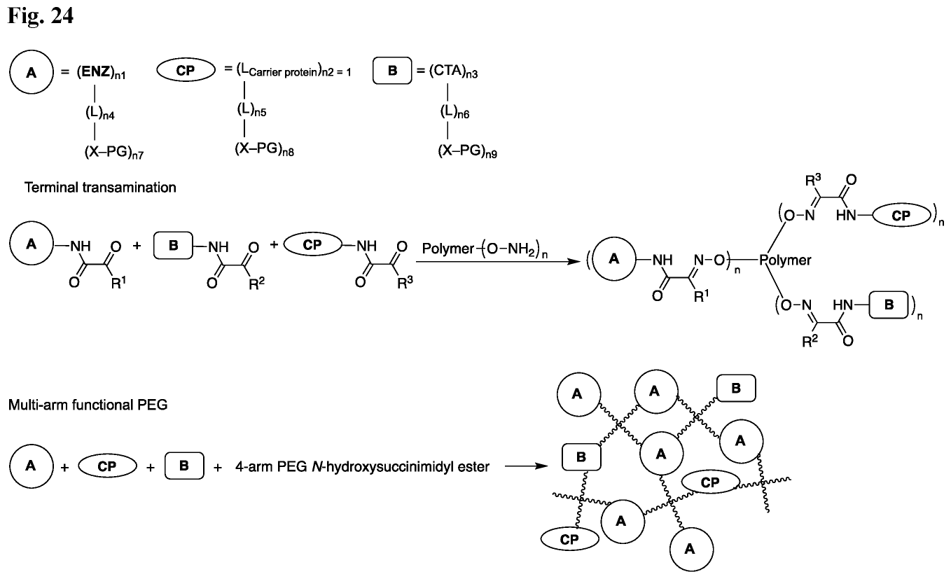

To construct Formula I, design 3, 4, 5, or 7, a carrier protein may be employed as L. The carrier protein may be derivatized with the ENZ or CTA by the method of terminal transamination or via use of a multi-arm PEG. Loading of L or X-PG units on the ENZ, carrier protein or CTA may be achieved prior to the joining reactions to assemble (ENZ)ₙ₁—(L)ₙ₂-(CTA)ₙ₃ of Formula I, design 3, 4, or 5. Alternatively, for Formula I, design 7, the L or X-PG units can be installed after the assembly of the constituents to give (ENZ)ₙ₁—Pₙ₂—(CTA)ₙ₃ (FIG. 24).

In some designs, it may be possible that the ENZ is joined to the CTA or a carrier protein without linkers. Bioengineering is a powerful tool to express fusion proteins, which can encompass ENZ-CTA or ENZ-carrier protein architectures. Bioengineering is also an optional tool to introduce or eliminate bioconjugatable sites (See above discussion on amine landscaping).

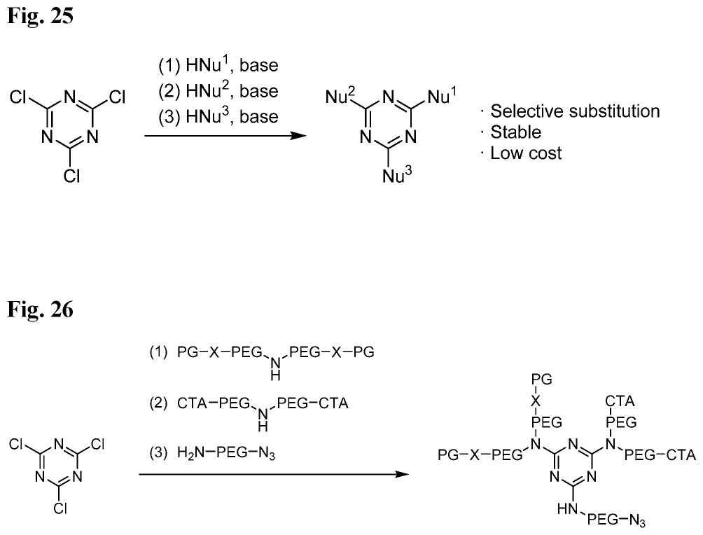

Selective and successive substitution of three chlorine atoms in 2,4,6-trichloro-1,3,5-triazine (cyanuric chloride) is well-known^{Afo} and is exemplified by triazine dendrimers^{Ste,Lee} (FIG. 25). The use of inexpensive cyanuric chloride can be advantageous in large-scale synthesis. Remarkably, such methods may be employed with little or no purification given the selectivity of single substitution. In other words, the first chloride is readily displaced; the second chloride is displaced with greater difficulty; and the third chloride requires the most forcing conditions for displacement. Hence, three successive substitutions may be carried out in a straightforward, non-statistical manner.

The properties of cyanuric chloride are suitable for rapid assembly of building blocks. FIG. 26 shows an example assembly of two PEG-X-PG moieties, two CTA-PEG moieties, and a PEG-azide moiety at the triazine core.

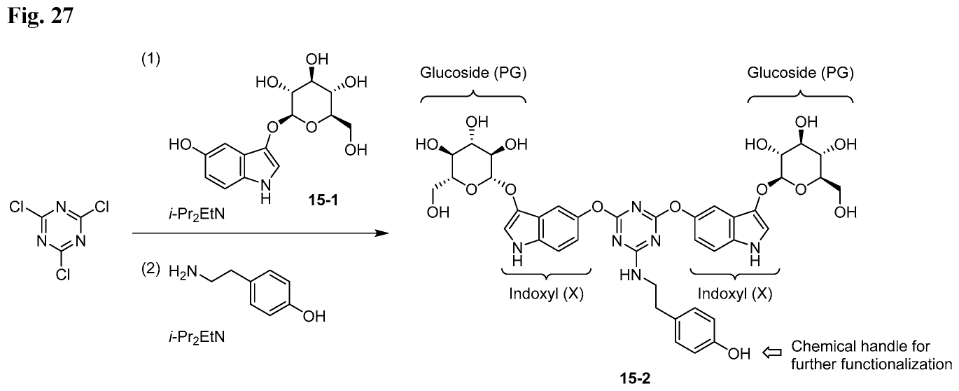

The triazine core can act as a branching unit if the two substituents at the triazine are identical. Compound 15-2 is an example of a triazine-based branching unit (FIG. 27). Two indoxyl glucosides as X-PGs are present, and a phenolic hydroxy group provides for further functionalization. The indoxyl glucoside 15-1 is prepared from the known acetyl-protected indoxyl glucoside.²⁰⁰⁶⁰¹⁰¹⁶⁵

The present invention is explained in greater detail in the following non-limiting examples.

### EXAMPLES

### Example 1

### Formula I, Design 1

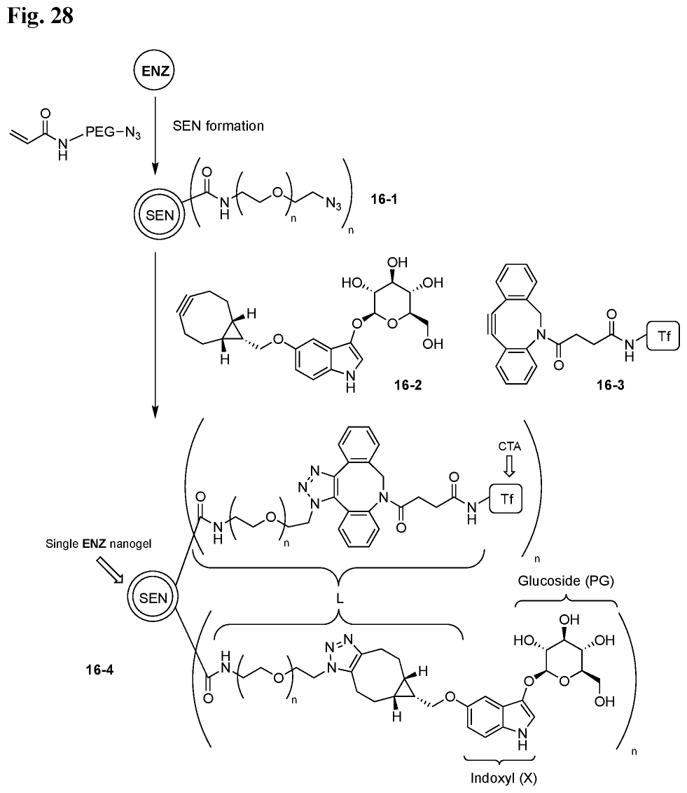

In this architecture, the ENZ is compartmentalized to give the SEN, which in so doing is derivatized with PEG units that are terminated with azide groups (16-1) (see method 2 in FIG. 18). A click reaction is carried out with two cycloalkynes to form the corresponding triazoles (16-4). One alkyne is attached to an indoxyl glucoside (16-2) as the X-PG unit whereas the other (16-3) is attached to the CTA (in this case, transferrin) (FIG. 28). Compound 16-2 is prepared by the O-alkylation of 15-1 to connect the cycloalkyne moiety. In this case, the relative loading of the X-PG and CTA units can be controlled by the ratios of the reactants. Note that here, the azide groups are introduced to the SEN before forming the ENZ-CTA linkage.

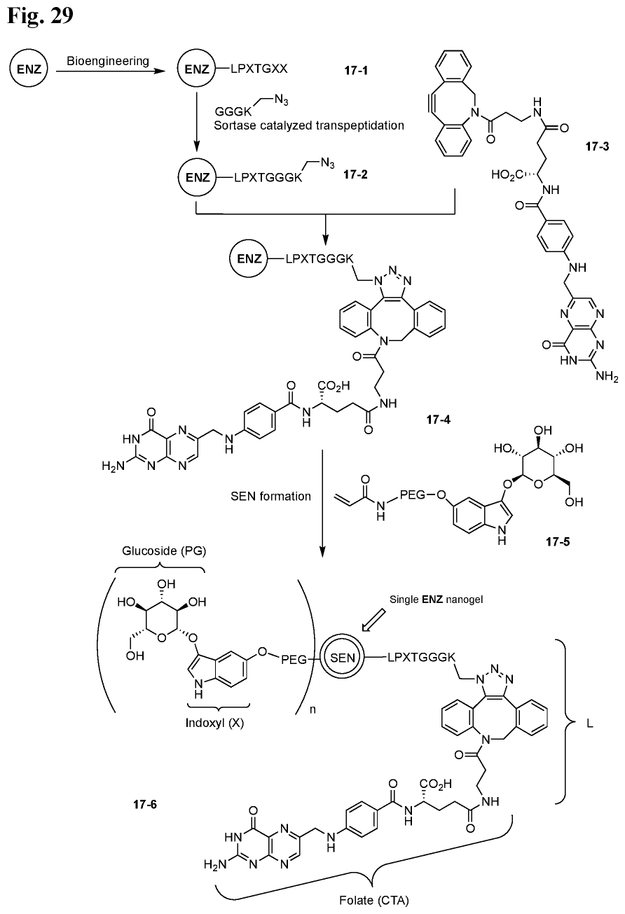

In FIG. 29, the ENZ is subjected to bioengineering to attach a peptide to the carboxylic terminus, forming 17-1. Sortase-catalyzed transpeptidation installs an azide-terminated lysine, forming 17-2. Reaction of 17-2 with a folate-cycloalkyne conjugate (17-3) affords the ENZ bearing a single folate unit (17-4). Folate is a small molecule that serves as the CTA. Here, the SEN is formed after the ENZ-CTA joining (see Method 1 in FIG. 19). SEN formation creates the compartmentalization of the ENZ and installs the PG-X groups (indoxyl-glucoside) to give 17-6, which bears a single folate unit (CTA). Acrylamide 17-5 is prepared by the acryloylation of the corresponding H₂N-PEG-indoxyl-glucoside, which can be synthesized from protected H₂N-PEG-OTs and 15-1.

### Example 2

### Formula I, Design 2

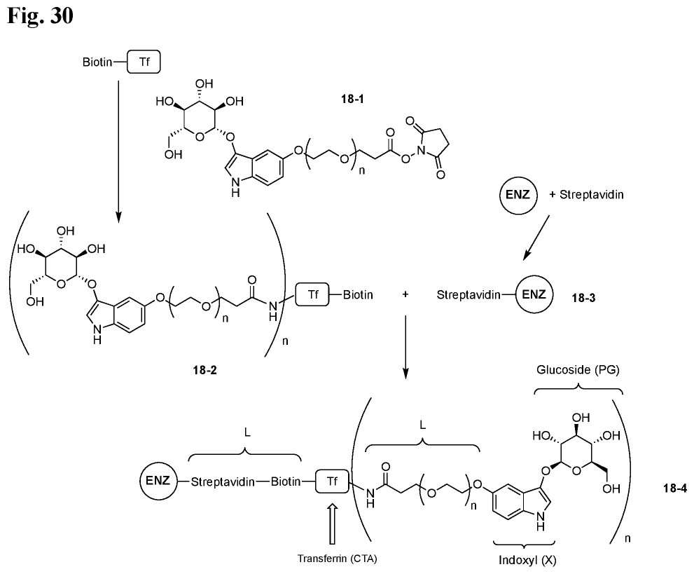

In FIG. 30, commercially available biotinylated transferrin (Biotin-Tf) serves as the CTA bearing half linker. Indoxyl glucoside (X-PG) bearing a PEG linker and N-hydroxysuccinimidyl ester (18-1), which is prepared by derivatization of 15-1 with a heterobifunctional PEG linker, is conjugated with transferrin to form 18-2. The ENZ bearing the other half linker (18-3) is prepared with a commercially available streptavidin. The biotin-streptavidin binding phenomenon has been exploited for use in many labeling applications. Here, the binding of biotin and streptavidin forms the design 18-4 (FIG. 30).

### Example 3

### Formula I, Design 3

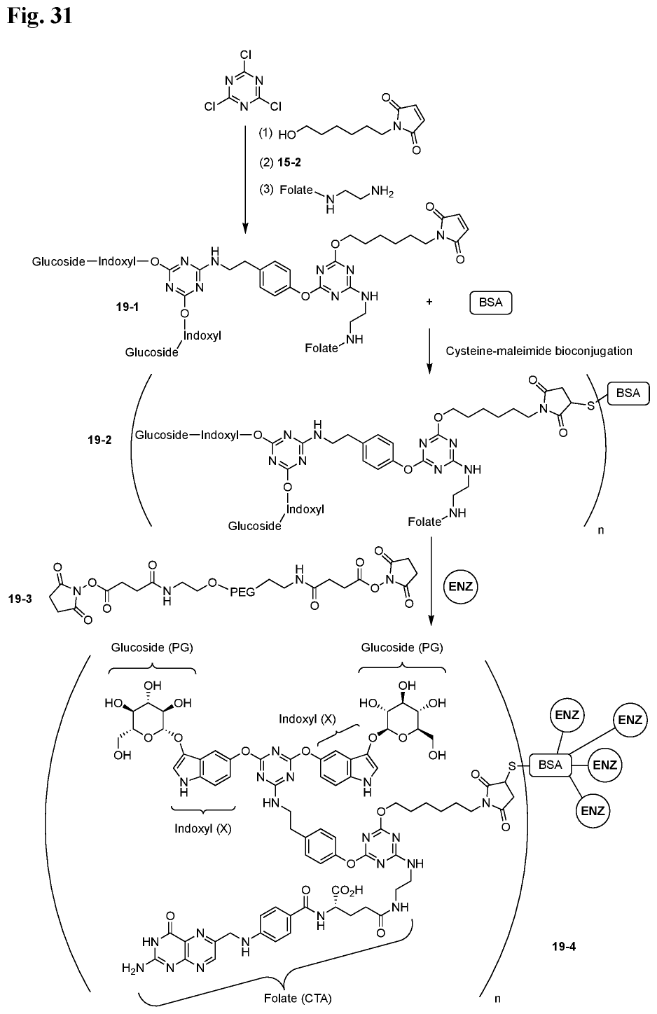

An example for Formula I, design 3 is shown in FIG. 31. A branched triazine linker is used to prepare 19-1 containing (3) folate, a small molecule CTA, (2) 15-2 bearing two indoxyl glucosides (X-PG), and (1) a maleimide group as a bioconjugatable group. This molecule can be derivatized with the cysteine groups on BSA, the carrier protein, forming 19-2. Afterwards, multiple ENZ constituents can be attached to 19-2 by reaction in the presence of a bifunctional PEG linker (19-3) to form 19-4, the example for Formula I, design 3. BSA self-coupling is possible but can be thwarted by use of excess quantities of the ENZ; any such self-coupling products can be removed by standard separation methods. The simplicity of this statistical approach has merit in enabling rapid assembly of a desirable target architecture.

### Example 4

### Formula II, Design 1

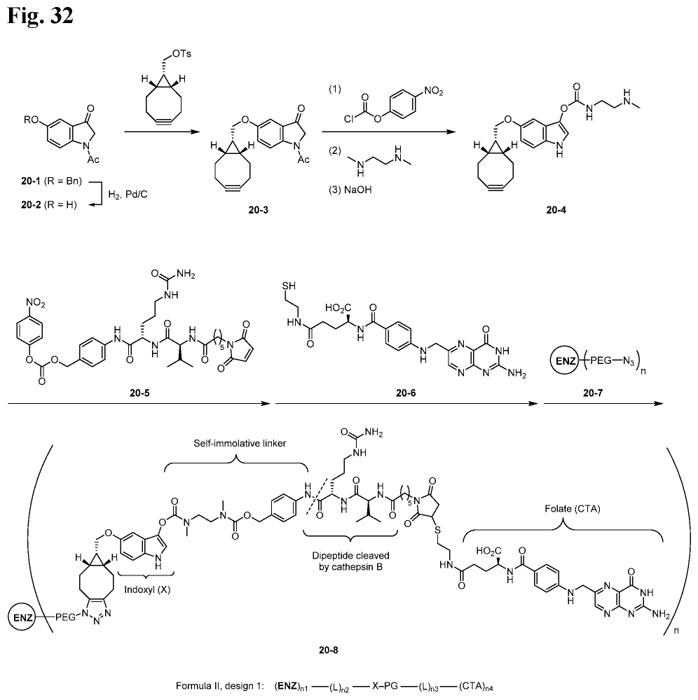

An example for Formula II, design 1 is shown in FIG. 32. The benzyl group of indanone 20-1 is deprotected by Pd/C catalysis to give 20-2, which upon reaction with bicyclononylmethyl tosylate gives 20-3. Three subsequent transformations afford amine 20-4, which is then linked with (i) the nitrophenyl carbonate-activated cathepsin B-labile dipeptide (20-5),^{Dubo} (ii) a thiol-bearing folate moiety as the CTA (20-6), and (iii) an ENZ bearing azide-terminated PEG linkers (20-7) via click chemistry to give the target example 20-8. Here the self-immolative linker and the cathepsin B-labile dipeptide together form the X-PG group in the backbone of Formula II. Cleavage by cathepsin B in lysosomes releases the ENZ bearing cross-linking groups for subsequent in vivo matrix formation.

### Example 5

### Formula II, Design 2

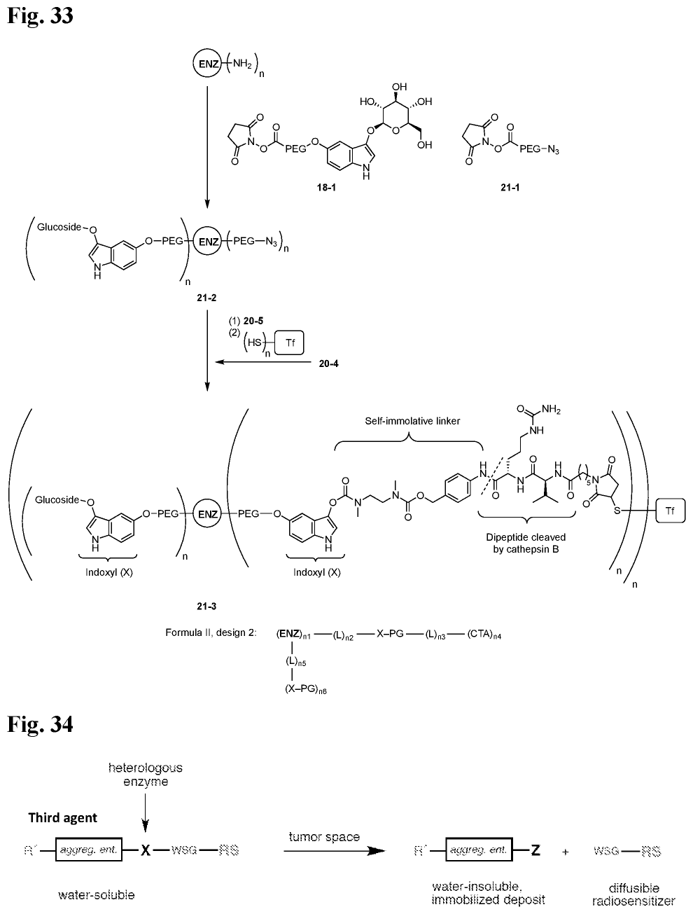

In construction of examples of this design, the lysine residues of the ENZ are derivatized with PEG-activated esters 18-1 and 21-1 in a statistical manner to form 21-2 (FIG. 33). Compound 18-1 has the indoxyl glucoside as X-PG whereas 21-1 has an azide terminus. The alkyne/azide click reaction of 21-2 with the conjugate 20-5 (prepared from 20-4) and transferrin affords the target compound 21-3. This is an example of Formula II, design 2.

### REFERENCES

Faul “Killing of Human Tumor Cells in Culture with Adriamycin Conjugates of Human Transferrin,” Yeh, C. -J. G.; Faulk, W. P. Clin. Immunol. Immunopathol. 1984, 32, 1-11

Fran1 “N-Terminal Protein Modification through a Biomimetic Transamination Reaction,” Gilmore, J. M.; Scheck, R. A.; Esser-Kahn, A. P.; Joshi, N. S.; Francis, M. B. Angew. Chem. Int. Ed. 2006, 45, 5307-5311.

Fran2 “Regioselective Labeling of Antibodies through N-Terminal Transamination,” Scheck, R. A.; Francis, M. B. ACS Chem. Biol. 2007, 2, 247-251.

Fran3 “Protein-Cross-Linked Polymeric Materials through Site-Selective Bio-Conjugtion,” Esser-Kahn, A. P.; Francis, M. B. Angew. Chem. Int. Ed. 2008, 47, 3751-3754.

Fran4 “Site-specific Protein Bioconjugation via a Pyridoxal 5′-Phosphate-Mediated N-Terminal Transamination Reaction,” Witus, L. S.; Francis, M. Curr. Protoc. Chem. Biol. 2010, 2, 125-134.

Fran5 “Targeting the N Terminus for Site-Selective Protein Modification,” Rosen C. B.; Francis, M. Nat. Chem. Biol. 2017, 13, 697-705.

Mann “Transferrin conjugation confers mucosal molecular targeting to a model HIV-1 trimeric gp140 vaccine antigen,” Mann, J. F. S.; Stieh, D.; Klein, K.; Miranda de Stegmann, D. S.; Cranage, M. P.; Shattock, R. J.; McKay P. F. J. Control. Release. 2012, 158, 240-249.

Nish1 “High-Throughput Protein Glycomics: Combined Use of Chemoselective Glycoblotting and MALDI-TOF/TOF Mass Spectrometry,” Nishimura, S. -I.; Niikura, K.; Kurogochi, M.; Matsushita, T.; Fumoto, M.; Hinou, H.; Kamitani, R.; Nakagawa, H.; Deguchi, K.; Miura, N.; Monde, K.; Kondo, H.; Angew. Chem. 2005, 117, 93-98.

Nish2 “Versatile Glycoblotting Nanoparticles for High-Throughput Protein Glycomics,” Niikura, K.; Kamitani, R.; Kurogochi, M.; Uematsu, R.; Shinohara, Y.; Nakagawa, H.; Deguchi, K.; Monde, K.; Kondo, H.; Nishimura, S. -I. Chem. Eur. 1 2005, 11, 3825-3834.

Pang “Conjugation Reaction with 8-Arm PEG Markedly Improves the Immunogenicity of Mycobacterium tuberculosis CFP10-TB10.4 Fusion Protein,” Sun, X.; Yu, W.; Pang, Q.; Hu, T. Bioconjugate Chem. 2017, 28, 1658-1668.

Ploe “Production of Unnaturally Linked Chimeric Proteins Using Combination of Sortase-Catalyzed Transpeptidation and Click Chemistry,” Witte, M. D.; Theile, C. S.; Wu, T.; Guimaraes, C. P.; Blom, A. E. M.; Ploegh, H. L. Nat. Protoc. 2013, 8, 1808-1819.

Pras “Bioconjugated PLGA-4-Arm-PEG Branched Polymeric Nanoparticles as Novel Tumor Targeting Carriers,” Ding, H.; Yong, K. -T.; Roy, I.; Hu, R.; W, F.; Zhao, L.; Law W. -C.; Zhao, W.; Ji, W.; Liu, L.; Bergey, E. J.; Prasad, N. Nanotechnology 2011, 22, 165101.

Qin “Targeted Drug Delivery via the Transferrin Receptor-Mediated Endocytosis Pathway,” Qin, Z. M.; Li, H.; Sun, H.; Ho, K. Parmacol. Rev. 2002, 54, 561-587.

Howa “Amine Landscaping to Maximize Protein-Dye Fluorescence and Ultrastable Protein-Ligand Interaction,” Jacobsen, M. T.; Fairhead, M; Fogelstrand, P.; Howarth, M. Cell Chem. Biol. 2017, 24, 1040-1047.

Herm “The reactions of Bioconjugation,” In Bioconjugate Techniques, Third Edition, Hermanson, G. T. Eds; Academic Press: San Diego, Calif., 2013, pp 229-258.

Vero “Peptide and protein PEGylation,” Veronese, F. M. Biomaterials 2001, 22, 405-417.

Rob “Chemistry for peptide and protein PEGylation,” Roberts, M. J.; Bentley, M. D.; Harris, J. M. Adv. Drug Deliv. Rev. 2002, 54, 459-476.

Koni “Developments and recent advancements in the field of endogenous amino acid selective bond forming reactions for bioconjugation,” Koniev, O.; Wagner, A. Chem. Soc. Rev. 2015, 44, 5495-5551.

Yan “Encapsulation of single enzyme in nanogel with enhanced biocatalytic activity and stability,” Yan, M.; Ge, J.; Liu, Z.; Ouyang, P. J. Am. Chem. Soc. 2006, 128, 11008-11009.

Belo “A simple route to highly active single-enzyme nanogels,” Beloqui, A.; Kobitski, A. Y.; Nienhaus, G. U.; Delaittre, G. Chem. Sci. 2018, 9, 1006-1013.

Gu “Protein Nanocapsule Weaved with Enzymatically Degradable Polymeric Network,” Gu, Z.; Yan, M.; Hu, B.; Joo, K. -I.; Biswas, A.; Huang, Y.; Lu, Y.; Wang, P.; Tang, Y. Nano Lett. 2009, 9, 4533-4538.

Afo “Synthesis of 2,4,6-Tri-substituted-1,3,5-Triazines,” Afonso, C. A. M.; Lourenco, N. M. T.; Rosatella, A. de A. Molecules 2006, 11, 81-102.

Ste “Dendrimers based on [1,3,5]-triazines,” Steffensen, M. B.; Hollink, E.; Kuschel, F.; Bauer, M.; Simanek, E. E. J. Polym. Sci. Part A Polym. Chem. 2006, 44, 3411-3433.

Lee “Functionalization of a Triazine Dendrimer Presenting Four Maleimides on the Periphery and a DOTA Group at the Core,” Lee, C.; Ji, K.; Simanek, E. Molecules 2016, 21, 335.

Dubo “Cathepsin B-Labile Dipeptide Linkers for Lysosomal Release of Doxorubicin from Internalizing Immunoconjugates: Model Studies of Enzymatic Drug Release and Antigen-Specific In Vitro Anticancer Activity,” Dubowchik, G. M.; Firestone, R. A.; Padilla, L; Willner, D; Hofstead, S. J.; Mosure, K.; Knipe, J. O.; Lasch, S. J.; Trail, P. A. Bioconjugate Chem. 2002, 13, 855-869.

2006010165 Mayers, G. L.; Lee, D; Chin, H. -L., Compositions and Methods for Treating Cancer. WO 2006010165 A2.

1. A compound comprising:
a cancer cell targeting agent;
a protecting group;
a cross-linking moiety; and
an enzyme.

2. The compound of claim 1, wherein the protecting group is an enzymatically cleavable protecting group.

3. The compound of claim 1, wherein the protecting group is directly attached to the enzyme or is attached to the enzyme via a linker.

4. The compound of claim 1, wherein the protecting group protects the enzyme from enzymatic degradation.

5. The compound of claim 1, wherein the protecting group is attached to the cross-linking moiety, thereby protecting the cross-linking moiety.

6. The compound of claim 1, wherein the compound has a structure represented by Formula I: 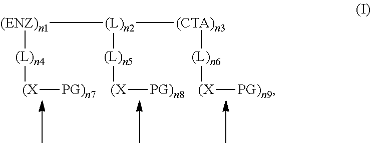
wherein CTA is the cancer cell targeting agent;
L are each an independently selected linking moiety;
PG are each an independently selected protecting group;
X are each an independently selected cross-linking moiety;
ENZ is the enzyme;
n1 and n3 are each independently an integer of 1 or 2 to 10, 50, or 100; and
n2, n4, n5, n6, n7, n8 and n9 are each independently an integer of 0 or 1 to 10, 50, or 100;
wherein the sum of n7, n8 and n9 is an integer of at least 1, at least 2, at least 10, or at least 20.

7. The compound of claim 1, wherein the compound has a structure represented by Formula II: 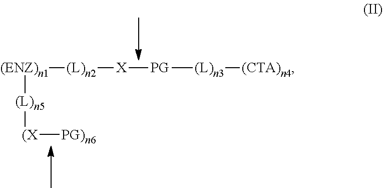
wherein CTA is the cancer cell targeting agent;
L are each an independently selected linking moiety that may be present or absent in the compound;
PG is the protecting group;
X is the cross-linking moiety;
ENZ is the enzyme; and
n1, n4, and n6 are each independently an integer of 1 or 2 to 10, 50, or 100; and
n2, n3, and n5 are each independently an integer of 0, 1, or 2 to 10, 50, or 100.

8. The compound of claim 1, further comprising a linker.

9. The compound of claim 1, wherein the protecting group is configured to be cleaved from the compound in vivo in a cell.

10. The compound of claim 1, wherein the cross-linking moiety is configured to cross-link in situ in a cell.

11. The compound of claim 1, wherein the compound comprises at least two protecting groups and at least two cross-linking moieties.

12. The compound of claim 1, wherein the cancer cell targeting agent binds to and/or targets an endocytosing receptor on a cell.

13. The compound of claim 1, wherein the enzyme is resistant to proteases and/or resistant to nucleases.

14. The compound of claim 1, further comprising one or more degradation shielding moieties.

15. The compound of claim 14, wherein the one or more degradation shielding moieties are selected from oligoethylene glycol groups and/or polyethylene glycol (PEG) groups.

16. (canceled)

17. The compound of claim 1, wherein the enzyme has activity toward a substrate that is not native in a cell.

18. The compound of claim 1, wherein the enzyme lacks activity toward native substrates in a cell.

19. The compound of claim 1, wherein the enzyme is heterologous to the subject.

20. A method of treating a subject having a solid tumor and/or reducing the size of a solid tumor in a subject, the method comprising:
administering a first agent comprising an enzyme to the subject;
administering a second agent to the subject, wherein the second agent comprises an anti-cancer agent; and
administering a radionuclide-derivatized compound to the subject, wherein the radionuclide-derivatized compound comprises a substrate for the enzyme, thereby treating the subject having the solid tumor and/or reducing the size the solid tumor in the subject.

21.-52. (canceled)

53. A method of treating a subject having a solid tumor and/or reducing the size a solid tumor in a subject, the method comprising:
localizing a first agent comprising an enzyme in a cancer cell in the subject;
releasing the enzyme from the cancer cell into the extracellular fluid; and
administering a radionuclide-derivatized compound to the subject, wherein the radionuclide-derivatized compound is converted by the enzyme from a soluble form to a less soluble form, thereby treating the subject having the solid tumor and/or reducing the size the solid tumor in the subject.

54. (canceled)
